(19) United States
(12) Patent Application Publication
(10) Pub. No.: US 2023/0270494 A1
(43) Pub. Date: Aug. 31, 2023

(54) TREATMENT APPARATUS FOR ENDOSCOPES AND EXPANDABLE FRAMES

(71) Applicant: HANGZHOU AGS MEDTECH CO., LTD., Hangzhou (CN)

(72) Inventors: Baiming SHI, Hangzhou (CN); Peng LI, Hangzhou (CN)

(73) Assignee: HANGZHOU AGS MEDTECH CO., LTD., Hangzhou (CN)

(21) Appl. No.: 18/314,155

(22) Filed: May 9, 2023

Related U.S. Application Data
(63) Continuation-in-part of application No. 16/518,987, filed on Jul. 23, 2019.
Application No. 16/518,987 is a continuation-in-part of application No. PCT/CN2018/071404, filed on Jan. 4, 2018.

(30) Foreign Application Priority Data
Jan. 23, 2017 (CN) 201710058825.2

(51) Int. Cl. A61B 18/14

(52) U.S. Cl. CPC A61B 18/1492; A61B 2018/126

(57) ABSTRACT

The present disclosure provides a treatment apparatus for an endoscope, comprising: a first electrode, the first electrode including an electrical treatment part and an operating wire; a sheath, a surface of the sheath being provided with a bendable flexible conductive part, the flexible conductive part being in the form of a tube, the flexible conductive part having a gap formed in the flexible conductive part to facilitate a bend of the flexible conductive part, the surface of the sheath being communicated with an external air of the flexible conductive part through the gap; wherein the operating wire is threaded within the sheath and the electrical treatment part extends out from a front end of the sheath.

### CROSS-REFERENCE TO RELATED APPLICATIONS

This application is a continuation in part of U.S. application Ser. No. 16/518,987, filed on Jul. 23, 2019, which is a continuation in part of International Application No. PCT/CN2018/071404, filed on Jan. 4, 2018, which claims priority to Chinese Patent Application No. 201710058825.2, filed on Jan. 23, 2017, the contents of each of which are hereby incorporated by reference in its entirety.

### BACKGROUND

### Technical Field

The present invention belongs to the field of medical instruments, and specifically, to a treatment apparatus for an endoscope and an expandable frame.

### Related Art

There are so far known bipolar treatment instruments for an endoscope that are passed through endoscopes to treat living tissue and the like. Some of the bipolar treatment instruments for an endoscope have, for example, a function of supplying a high-frequency current to a treatment instrument to perform treatments such as incision, cauterization, and hemostasis on living tissue.

As an example of such a treatment instrument for an endoscope, a high-frequency incision instrument that uses a polypectomy snare supplied with a high-frequency current to incise living tissue is recorded in Patent CN102413786B. An electric wire, a passive electrode arranged on a periphery of an extending part, living tissue in contact with the passive electrode, the polypectomy snare, and an operating wire are connected to a feeding electrode to form a current loop to excise a polyp. In addition, a bipolar sphincterotome is recorded in Patent CN205697995U and includes a control part, an insulating sheath, and a cutting part. A distal end of the control part is connected to a proximal end of the insulating sheath. The cutting part includes a cutting electrode, a passive electrode, a cutting electrode wiring terminal that may be connected to the cutting electrode, a passive electrode wiring terminal that may be connected to the passive electrode, a cutting electrode conductor connecting the cutting electrode and the cutting electrode wiring terminal, and a passive electrode conductor connecting the passive electrode and the passive electrode wiring terminal. The cutting electrode and the passive electrode are provided on a distal end of the insulating sheath and are respectively connected to the control part through the cutting electrode conductor and the passive electrode conductor.

However, both the foregoing technical solutions can be improved. First, an active electrode (that is, the foregoing high-frequency treatment part and cutting part) does not have an obvious thermal effect, and a cutting speed is relatively slow, resulting in limited clinical application. Second, an area of contact between a passive electrode and tissue is relatively small, and tissue may be burned accidentally. Third, in both the solutions, the passive electrode and the active electrode are relatively close, the two electrodes may contact or a current may flow through tissue fluids between the electrodes to cause a short circuit failure. Fourth, in a surgical instrument for an endoscope, treatment instruments such as electrocoagulation forceps, biopsy forceps, needle knives, and the like in addition to snares and papilla incision knives need to be electrified to perform incision, cauterization, hemostasis, among other work. The passive electrode in the foregoing technical solution has relatively low adaptability.

Endoscopic surgeries have been widely promoted and used in recent years due to the advantages of less incisional damage and better clinical results. In the endoscopic surgeries, a treatment apparatus for an endoscope with an electrical treatment part (e.g., an electric trap, etc.) is used to cut, cauterize, and stop the bleeding of biological tissues. At present, the treatment apparatus for an endoscope mainly includes a single electrode treatment apparatus and/or a double electrode treatment apparatus. Since the single electrode treatment apparatus for the endoscope has a wide range of a current with a relatively high frequency flowing through tissue(s) when it is used, which is more damaging to the tissue(s), the double electrode treatment apparatus for the endoscope has developed rapidly in recent years.

The existing double electrode treatment apparatus for the endoscope generally has a layer of metal electrode on a surface of a sheath as a return electrode, which is equivalent to an electrode sheet attached to the tissue(s), thus making the double electrode treatment apparatus for the endoscope have all the basic functions of the single electrode treatment apparatus for the endoscope, and also have a function of free switching between a single electrode mode (i.e., used as the single electrode treatment apparatus) and a double electrode mode (i.e., used as the double electrode treatment apparatus). However, if a length of the metal electrode on the surface of the sheath of the double electrode endoscope is set short, there may be problems such as poor conducting stability, poor conductivity and tissue damage; if it is set too long, it may affect the flexibility of the sheath, which is not conducive to a free movement of the sheath in an instrument passage of the endoscope with various curved angles, and it may affect the convenience of an operator. Therefore, it is desired to provide a treatment apparatus for an endoscope and an expandable frame to at least partially solve the above problems.

### SUMMARY

Based on this, to overcome the disadvantages in the prior art, the present invention provides a treatment apparatus for an endoscope, an endoscope, and an expandable frame. The structural design is appropriate, the cutting speed is relatively high, the risk of passive electrode burns is relatively low, and adaptability is provided.

The technical solutions of the present invention are as follows:

A treatment apparatus for an endoscope, comprising: a first electrode, comprising an electrical treatment part and an operating wire; a second electrode, configured to be installed on the endoscope, the second electrode comprising a first electrically conductive part and a sliding contact part electrically connected to the first electrically conductive part, and the first electrically conductive part being configured to contact a human body; a sheath, provided on a surface thereof with a second electrically conductive part, wherein the operating wire is passed through the sheath, the sheath is configured to be passed through the endoscope, and when the sheath is located in a preset position, the sliding contact part contacts and is electrically connected to the second electrically conductive part and the sheath is in sliding fit with the sliding contact part.

In one of the embodiments, further comprising an expandable frame, wherein the expandable frame is configured to be sleeved over the endoscope, the first electrically conductive part is provided outside the expandable frame, the first electrically conductive part is provided on the expandable frame, and a distance between a part, being configured to contact a human body, of the first electrically conductive part and an axis of the endoscope is greater than a radius of the endoscope.

In one of the embodiments, wherein a conductive material is provided on a peripheral surface of the expandable frame, and the conductive material forms the first electrically conductive part.

In one of the embodiments, wherein the expandable frame is silicone rubber that contains several conductive particles, and the conductive particles form the first electrically conductive part.

In one of the embodiments, wherein a layer of a conductive material is provided on an outer surface of the sheath, and the conductive material forms the second electrically conductive part.

In one of the embodiments, wherein a first cavity and a second cavity are formed in the sheath, the operating wire is passed through the first cavity, a third electrically conductive part is provided in the second cavity, and the third electrically conductive part is electrically connected to the second electrically conductive part.

In one of the embodiments, wherein the second electrically conductive part surrounds the sheath by one loop in a circumferential direction of the sheath.

In one of the embodiments, wherein the sliding contact part comprises a fifth electrically conductive part and a sixth electrically conductive part, the sixth electrically conductive part is electrically connected to the first electrically conductive part, and the fifth electrically conductive part is electrically connected to the sixth electrically conductive part.

In one of the embodiments, wherein the expandable frame comprises a sleeve and a flexible part or an elastic part connected to the sleeve, the sleeve is configured to be sleeved over the endoscope, and the sliding contact part is fixed on an inner side of the sleeve.

A treatment apparatus for an endoscope, comprising: a first electrode, comprising an electrical treatment part and an operating wire; and a sheath, provided with a second electrically conductive part on an outer surface thereof, and the second electrically conductive part being configured to be electrically connected to a first electrically conductive part on an endoscope, wherein a first cavity and a second cavity are formed in the sheath, the operating wire is passed through the first cavity, a third electrically conductive part is provided in the second cavity, and the third electrically conductive part is electrically connected to the second electrically conductive part.

In one of the embodiments, wherein the second electrically conductive part is provided on an outer surface of the sheath.

In one of the embodiments, wherein a layer of a conductive material is provided on an outer surface of the sheath, and the conductive material forms the second electrically conductive part.

In one of the embodiments, wherein the second electrically conductive part surrounds the sheath by one loop in a circumferential direction of the sheath.

An expandable frame, wherein the expandable frame is configured to be installed on an endoscope, a diameter of a peripheral surface of the expandable frame is greater than a diameter of a peripheral surface of the endoscope, a first electrically conductive part and a sliding contact part electrically connected to the first electrically conductive part are provided on the expandable frame, the first electrically conductive part is provided on a periphery of the expandable frame to contact human body tissue, and the sliding contact part contacts and is electrically connected to a second electrically conductive part of the treatment apparatus for an endoscope in any one of the foregoing embodiments.

In one of the embodiments, wherein a conductive material is provided on the peripheral surface of the expandable frame, and the conductive material forms the second electrically conductive part.

In one of the embodiments, wherein the expandable frame comprises a sleeve and a transparent cover connected to the sleeve, the sleeve is configured to be sleeved over the endoscope, the transparent cover is transparent or translucent, and the transparent cover is configured to allow the endoscope to obtain an image.

In one of the embodiments, wherein the expandable frame comprises a sleeve and a flexible part or an elastic part connected to the sleeve, the sleeve is configured to be sleeved over the endoscope, and the sliding contact part is fixed on an inner side of the expandable frame.

An endoscope, comprising: a first electrically conductive part, provided on an outer surface of an endoscope, the first electrically conductive part being configured to contact a human body, an instrument passage being provided in the endoscope, the instrument passage being configured to be passed through by a treatment apparatus for an endoscope, a fourth electrically conductive part being provided inside the endoscope, and the first electrically conductive part being electrically connected to the fourth electrically conductive part.

In one of the embodiments, further comprising an expandable frame, wherein the expandable frame is installed outside the endoscope, and the first electrically conductive part is provided on the expandable frame.

In one of the embodiments, further comprising a tissue operation mechanism, wherein the first electrically conductive part and the fourth electrically conductive part are provided on the tissue operation mechanism, and the tissue operation mechanism is installed on the expandable frame.

In one of the embodiments, wherein two first electrically conductive parts are provided, one first electrically conductive part is provided on the tissue operation mechanism, the other first electrically conductive part is provided on the expandable frame, and the first electrically conductive part of the tissue operation mechanism is electrically connected to the first electrically conductive part of the expandable frame. In one of the embodiments, wherein a layer of a conductive material is provided on a peripheral surface of the endoscope, and the conductive material forms the first electrically conductive part.

In one of the embodiments, wherein a connecting through hole is formed in the endoscope, the peripheral surface of the endoscope and the instrument passage are communicated through the connecting through hole, and the conductive material is provided in the connecting through hole to electrically connect the first electrically conductive part to the fourth electrically conductive part.

In one of the embodiments, wherein the conductive material is provided on an end surface at a distal end of the endoscope to electrically connect the first electrically conductive part to the fourth electrically conductive part.

In one of the embodiments, further comprising a tissue operation mechanism, wherein the first electrically conductive part and the fourth electrically conductive part are provided on the tissue operation mechanism.

An expandable frame, configured to be installed outside an endoscope, a first electrically conductive part and a fourth electrically conductive part electrically connected to the first electrically conductive part being provided on the expandable frame, the first electrically conductive part being configured to contact a human body, and the fourth electrically conductive part being configured to be electrically connected to a feeding electrode.

In one of the embodiments, wherein the fourth electrically conductive part is a conductor wire, and the fourth electrically conductive part is configured to be passed through the endoscope or provided outside the endoscope.

The beneficial effects of the present invention are as follows:

The first electrode performs an electrical operation on a human body, and a current that enters the human body is looped through the second electrically conductive part instead of flowing all over the human body, so as to avoid damage to other organs or electronic apparatuses (for example, a pacemaker) in the human body, thereby ensuring the safety of the entire operation. Provided that electrical requirements can be satisfied, the second electrode may be provided at any position in the endoscope, so that a sufficiently large area of contact between the second electrically conductive part and the human body is ensured to fully guide out a current. A space of contact between an outer side of the endoscope body of the endoscope and tissue is fully used to increase a conductive area of a contact part through which a current is guided out from human body tissue in a return path, so that while the risk of burns is reduced, a thermal effect is further improved, thereby improving the security and operation efficiency of endoscopic surgery, and ensuring further clinical popularization and application of the treatment apparatus for an endoscope.

One or more embodiments of the present disclosure provide a treatment apparatus for an endoscope, comprising: a first electrode, the first electrode including an electrical treatment part and an operating wire a sheath, a surface of the sheath being provided with a bendable flexible conductive part, the flexible conductive part being in the form of a tube, the flexible conductive part having a gap formed in the flexible conductive part to facilitate a bend of the flexible conductive part, the surface of the sheath being communicated with an external air of the flexible conductive part through the gap; wherein the operating wire is threaded into the sheath and the electrical treatment part extends from a front end of the sheath.

In some embodiments, the flexible conductive part includes a metal member containing the gap, the metal member including one or more consecutive metal members, the gap between the one or more consecutive metal members being formed such that the surface of the sheath is partially exposed.

In some embodiments, the gap includes a slit formed between interrupted surfaces of the one or more consecutive metal members, the slit being spirally provided in an axial direction of the sheath.

In some embodiments, a pitch of the spirally provided slit is 0.1 to 2 mm.

In some embodiments, a width of the gap along the axial direction of the sheath is less than a length of a conductive structure cooperating with the flexible conductive part extending along an axial direction of the sheath.

In some embodiments, the flexible conductive part includes a metal member containing the gap, a width of the gap along an axial direction of the sheath being greater than or equal to a wall thickness of the metal member.

In some embodiments, the flexible conductive part includes a bent section and a straight section, the straight section being electrically connected to the bent section, a length of the straight section being less than or equal to six times a diameter of the straight section.

In some embodiments, the flexible conductive part includes two straight sections, the two straight sections including the straight section and a second section, the bent section being connected between the two straight sections, a length of the bent section being greater than or equal to two times the diameter of the bent section.

In some embodiments, the front end of the sheath extends out from a front end of the flexible conductive part.

In some embodiments, a first distance between the front end of the flexible conductive part and the front end of the sheath is in a range from 0.5 mm to 1.5 mm.

In some embodiments, the treatment apparatus for an endoscope further includes a second electrode and an expandable frame, wherein the second electrode is used to be mounted on the endoscope, the second electrode includes a first conductive part and a sliding contact part electrically connected to the first conductive part, the expandable frame is used to fit on an outside of the endoscope, the first conductive part is provided on a periphery of the expandable frame; the sheath is used to be threaded inside the endoscope, the sliding contact part is in contact with and electrically connected to the flexible conductive part when the sheath is in a preset position.

In some embodiments, the first conductive part includes a metal conductive layer of a variable diameter provided on the periphery of the expandable frame.

In some embodiments, the metal conductive layer is in a bow shape.

In some embodiments, the sliding contact part includes a metal shrapnel protruding inwardly, the metal shrapnel being electrically connected to the first conductive part; the metal shrapnel is provided with two lugs on both sides of the metal shrapnel, the two lugs being electrically connected to the metal shrapnel, the two lugs and the metal shrapnel forming a semi-enveloping structure for the flexible conductive part.

In some embodiments, the sliding contact part is provided on the expandable frame, the expandable frame includes a transparent straight frame provided at a front end of the expandable frame, and a front end of the sliding contact part has a second distance from a front end of the transparent frame.

In some embodiments, the second distance is in a range from 0.5 mm to 1.5 mm.

In some embodiments, a first distance between the front end of the flexible conductive part and the front end of the sheath is equal to the second distance.

In some embodiments, the sheath is provided with a return conductor, the return conductor passing through the sheath and being electrically connected to the flexible conductive part; the sheath is provided with an opening through which the return conductor passes, the opening being provided with a metal restriction tube, and the flexible conductive part being connected to the metal restriction tube.

One or more embodiments of the present disclosure provide a treatment apparatus for an endoscope, comprising: a first electrode, the first electrode including an electrical treatment part and an operating wire; a sheath, a surface of the sheath being provided with a bendable flexible conductive part; wherein the operating wire is threaded into the sheath, the electrical treatment part extends out from a front end of the sheath, and the front end of the sheath extends out from a front end of the flexible conductive part.

One or more embodiments of the present disclosure provide an expandable frame, wherein the expandable frame is provided with a first conductive part on a periphery of the endoscope, the first conductive part including a metal conductive layer of a variable diameter.

### BRIEF DESCRIPTION OF THE DRAWINGS

FIG. 1A is a schematic diagram of coordination between a treatment apparatus for an endoscope according to Embodiment 1 of the present invention and an endoscope;

FIG. 1B is a schematic diagram of coordination between a treatment apparatus for an endoscope according to Embodiment 1 of the present invention and an endoscope;

FIG. 2 is a schematic diagram of an expandable frame installed in a treatment apparatus for an endoscope according to Embodiment 1 of the present invention;

FIG. 3 is a schematic diagram of an end surface in FIG. 2;

FIG. 4 is a diagram showing several arrangement manners of a second conductive body according to Embodiment 1 of the present invention;

FIG. 5A to FIG. 5C are schematic diagrams of other implementations of the expandable frame according to Embodiment 1 of the present invention and an endoscope;

FIG. 6A to FIG. 6C are schematic diagrams of coordination between a treatment apparatus for an endoscope according to Embodiment 2 of the present invention and an endoscope;

FIG. 7A to FIG. 13 are schematic diagrams according to another implementation of the present invention;

FIG. 14 is a schematic diagram according to another implementation of the present invention;

FIG. 15 is an enlarged view of A in FIG. 14;

FIG. 16 is a schematic diagram according to another implementation of the present invention;

FIG. 17 is an enlarged view of B in FIG. 16;

FIG. 18 is a schematic diagram according to another implementation of the present invention;

FIG. 19 is an enlarged view of C in FIG. 18;

FIG. 20 is a schematic diagram according to another implementation of the present invention;

FIG. 21 is an enlarged view of E in FIG. 20;

FIG. 22 is a schematic diagram according to Embodiment 3 of the present invention;

FIG. 23 is a partial enlarged view of FIG. 22;

FIG. 24 is a schematic diagram according to another implementation of the present invention;

FIG. 25 is a partial sectional view of FIG. 24;

FIG. 26 is a schematic diagram of an overall structure of the implementation in FIG. 24;

FIG. 27 is a structural diagram 1 according to Embodiment 4 of the present invention;

FIG. 28 is a partial enlarged view of a circle in FIG. 24.

FIG. 29 is a structural diagram 2 according to Embodiment 4 of the present invention; and

FIG. 30 is a structural diagram 3 according to Embodiment 4 of the present invention.

FIG. 31 is a schematic diagram illustrating an exemplary structure of a treatment apparatus for an endoscope according to some embodiments of the present disclosure;

FIG. 32 is a schematic diagram illustrating an exemplary structure of a treatment apparatus for an endoscope cooperating with an endoscope according to some embodiments of the present disclosure;

FIG. 33 is a schematic diagram illustrating an exemplary structure of an expandable frame mounted on the treatment apparatus for an endoscope according to some embodiments of the present disclosure;

FIG. 34 is a schematic diagram illustrating an exemplary structure of an expandable frame and a sheath cooperating with an endoscope, according to some embodiments of the present disclosure;

FIG. 35 is a schematic diagram illustrating another exemplary structure of an expandable frame and a sheath cooperating with an endoscope, according to some embodiments of the present disclosure;

FIG. 36 is a schematic diagram illustrating an exemplary working simulation of an expandable frame and a sheath cooperating with an endoscope according to some embodiments of the present disclosure;

FIG. 37 is a schematic diagram illustrating an exemplary application of a flexible conductive part according to some embodiments of the present disclosure;

FIG. 38 is a schematic diagram illustrating an exemplary structure of a bent section of a flexible conductive part according to some embodiments of the present disclosure;

FIG. 39 is a schematic diagram illustrating an exemplary structure of a flexible conductive part according to some embodiments of the present disclosure;

FIG. 40 is a schematic diagram illustrating an exemplary structure of an expandable frame and a first conductive part cooperating with a sliding contact part according to some embodiments of the present disclosure;

FIG. 41 is a schematic diagram illustrating another exemplary structure of an expandable frame and a first conductive part cooperating with a sliding contact part according to some other embodiments of the present disclosure;

FIG. 42 is a schematic diagram illustrating an exemplary structure of a metal conductive layer according to some embodiments of the present disclosure; and

FIG. 43 is a cross-sectional diagram illustrating a sheath and a flexible conductive part according to some embodiments of the present disclosure.

### REFERENCE NUMERALS

Sheath 10, first cavity 11, second cavity 12, operating wire 20, cutting part 21, ESD knife 222, electric snare 221, electrocoagulation forceps 223, electric biopsy forceps 224, conductor cavity 2251, cutting wire 2252, anchor 2253, drive part 30, sliding ring 31, endoscope body 401, instrument passage 403, transparent cover 41, expandable frame 42, return path 50, first electrically conductive part 51, second electrically conductive part 52, sliding contact part 53, third electrically conductive part 54, fourth electrically conductive part 55, feeding electrode 60, passive electrode 61, active electrode 62, tissue operation mechanism 70, treatment apparatus 100, first electrode 110, electrical treatment part 111, operating wire 112, sheath 120, flexible conductive part 121, return conductor 122, metal restriction tube 123, second electrode 130, first conductive part 131, sliding contact part 132, metal conductive layer 1311, metal shrapnel 1321, lug 1322, expandable frame 140, transparent straight frame 141, drive part 150, sliding ring 151, endoscope 160, instrument passage 161, lens 162.

### DETAILED DESCRIPTION

The present invention is further described below in detail, but the implementations of the present invention are not limited thereto.

### Embodiment 1

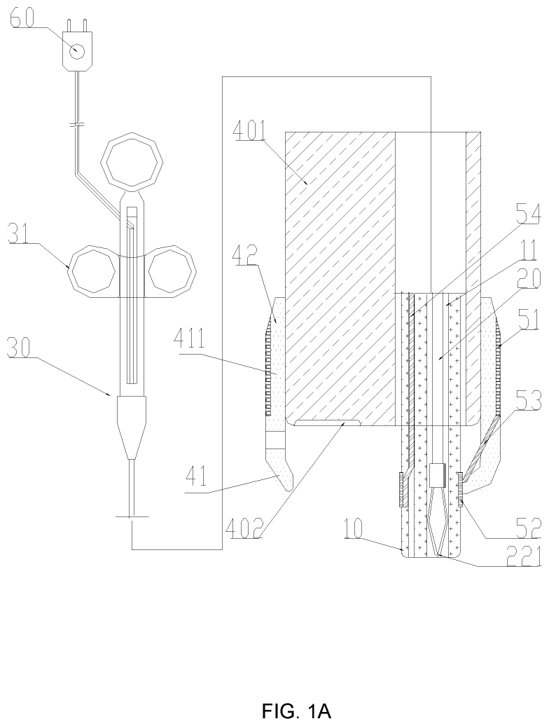

As shown in FIG. 1A to FIG. 3, a treatment apparatus for an endoscope includes: a first electrode, including an electrical treatment part and an operating wire 20; a second electrode, configured to be provided inside the endoscope (not shown), and including a first electrically conductive part 51 and a sliding contact part 53 electrically connected to the first electrically conductive part 51, the first electrically conductive part 51 being configured to contact a human body; and a sheath 10, provided on a surface thereof with a second electrically conductive part 52, where the operating wire 20 is passed through the sheath 10, the sheath 10 is passed through the endoscope, and the sheath 10 is movable back and forth in an instrument passage of the endoscope. Specifically, the sheath 10 of the present invention is passed through the instrument passage 403 of the endoscope and is controlled by a drive part 30 (a sliding ring 31 is provided on the drive part 30) connected outside a body to move freely forward or backward in the instrument passage 403.

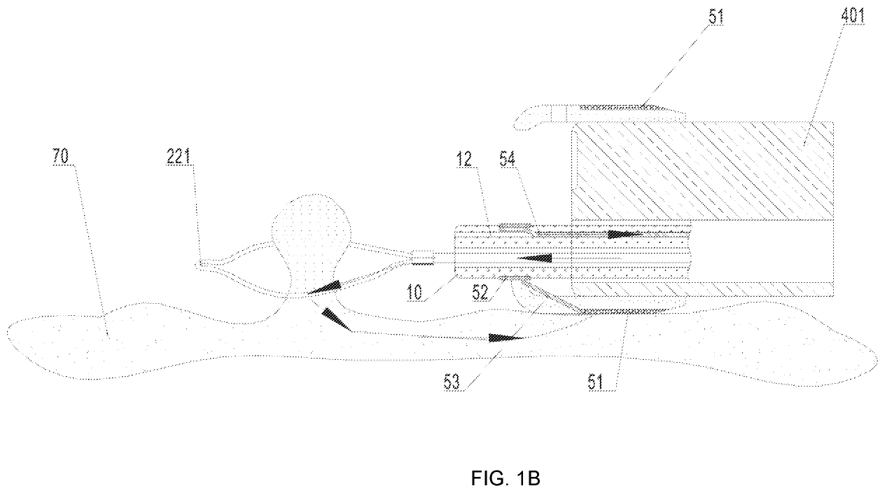

As shown in FIG. 2, a first cavity 11 extending in an axial direction and having openings at two ends is formed in the sheath 10. The sheath 10 is made of an insulating material, for example, insulating resin. In addition, preferably, the sheath 10 needs to be flexible enough to bend and move forward or backward along lumen tissue and the like in an organism.

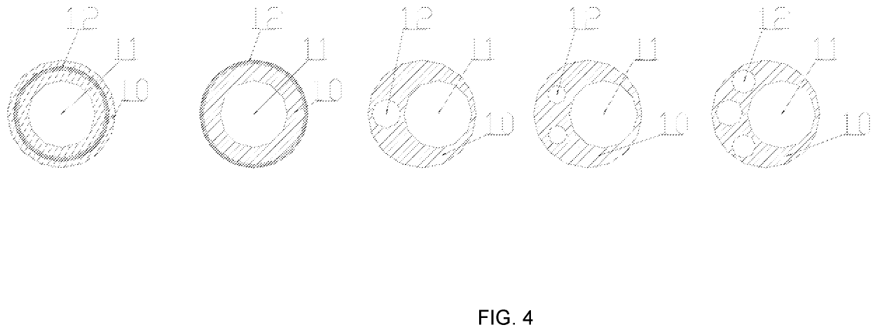

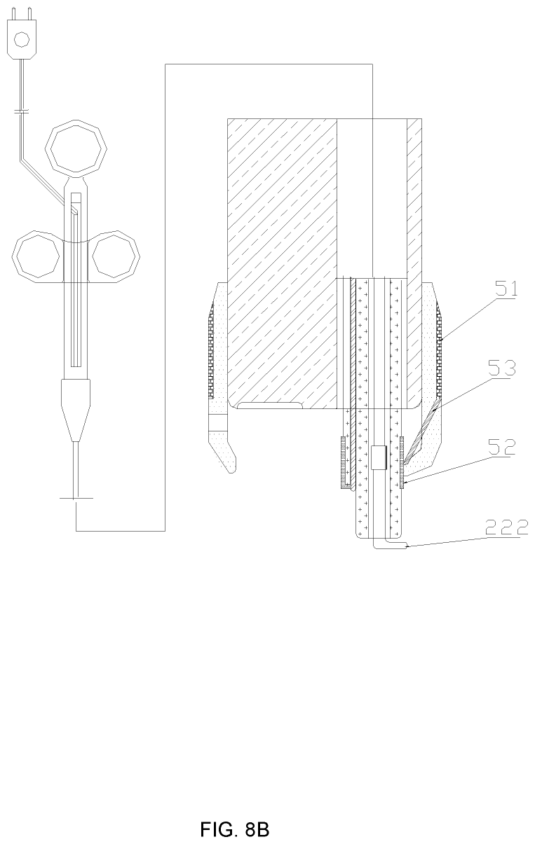

As shown in FIG. 1A, FIG. 1B, and FIG. 4, the first cavity 11 and a second cavity 12 are formed in the sheath 10. The conductive operating wire 20 is passed through the first cavity 11 in a manner of moving freely forward or backward. The electrical treatment part (including, but is not limited to, an endoscopic submucosal dissection (ESD) knife 222, an electric snare 221, electrocoagulation forceps 223, electric biopsy forceps 224, and a papilla sphincterotomy hereinafter) is provided at a distal end of the operating wire 20, and in this embodiment, is an electric snare. The operating wire 20 is conductive. A proximal end of the operating wire 20 is electrically connected to an active electrode 62 of a feeding electrode 60 (in the present invention, the feeding electrode 60 includes a passive electrode 61 and the active electrode 62, referring to FIG. 14 for details). Although the operating wire 20 is conductive (or even the operating wire 20 and the electrical treatment part may be one overall conductor) in this embodiment, the specific material and performance of the operating wire 20 are not limited, provided that the electrical treatment part can be manipulated to move forward or backward. Alternatively, the operating wire 20 may be not conductive, and a conductor wire is additionally used between the electrical treatment part and an electrode to establish a conductive connection.

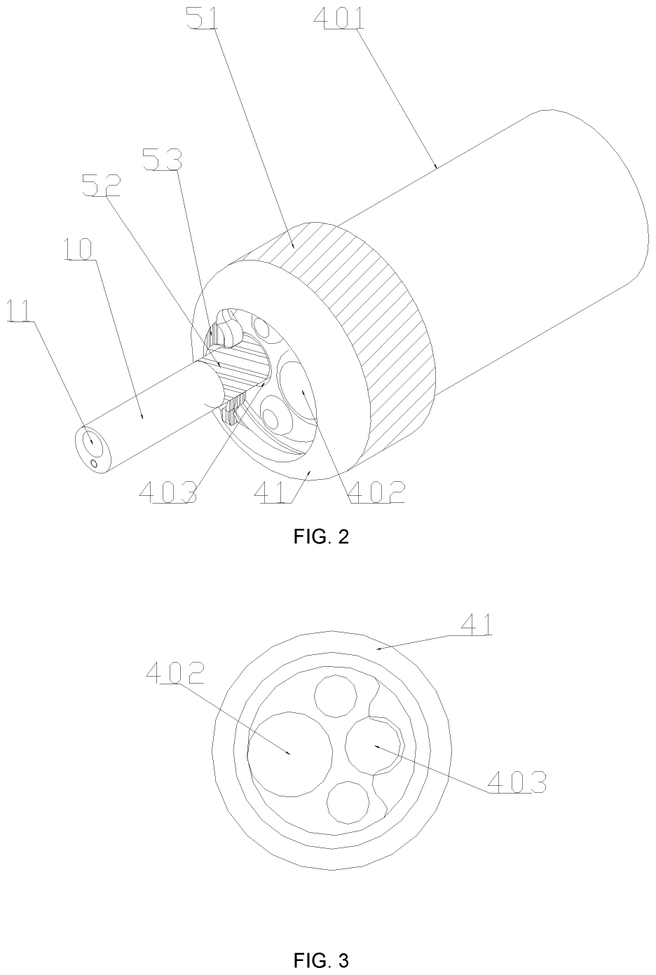

An expandable frame is sleeved over a front end of the endoscope. As shown in FIG. 2 and FIG. 3. The expandable frame 42 includes a sleeve 411 sleeved over the endoscope and a transparent cover 41. The transparent cover 41 is made of a transparent insulating material. The transparent cover 41 may block a part of the view of a camera 402 (as shown in FIG. 1 and FIG. 2) of the endoscope, and the endoscope may obtain an image through the transparent cover 41. The first electrically conductive part 51 is provided on a periphery of the expandable frame. A diameter of a peripheral surface of the expandable frame is greater than a diameter of a peripheral surface of the endoscope. The sliding contact part 53 may be provided on the expandable frame.

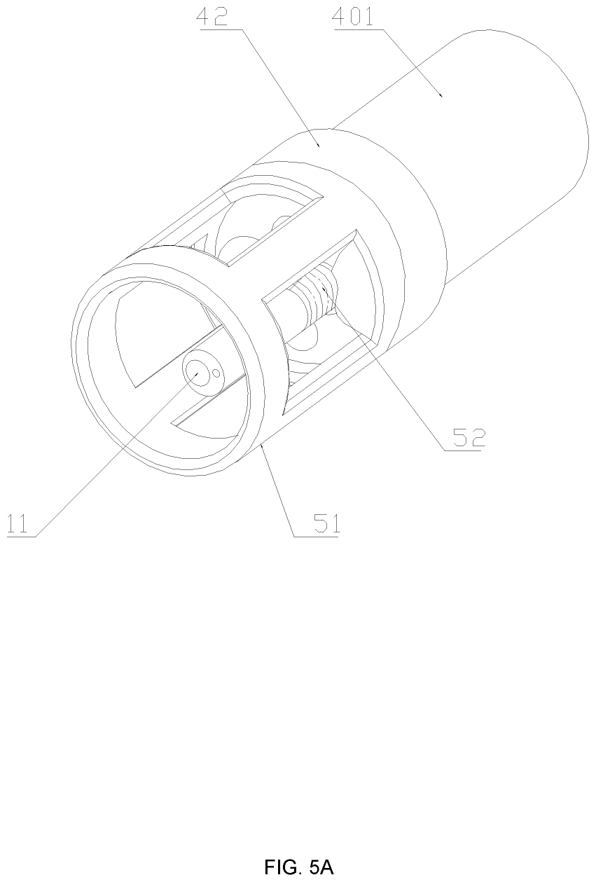

FIG. 5A, FIG. 5B, and FIG. 5C further show other implementations in which the expandable frame 42 is installed on the treatment apparatus for an endoscope. During use, the expandable frame 42 is assembled at a distal end of an endoscope body 401 of the endoscope, the first electrically conductive part 51 is provided on a periphery of the expandable frame, and the first electrically conductive part 51 has a relatively large surface area to fully contact human body tissue. Preferably, a structure of the first electrically conductive part 51 may be as follows: A conductive material is provided on a peripheral surface of the endoscope 42, and the conductive material forms the first electrically conductive part 51. The type of the conductive material may be selected as required. In addition, preferably, the sliding contact part 53 is provided on the expandable frame 42 and has conductive performance. A part of the sliding contact part 53 is electrically connected to the first electrically conductive part 51, and another part of the sliding contact part 53 forms tight fit with a periphery of the sheath and is electrically connected to the second electrically conductive part 52.

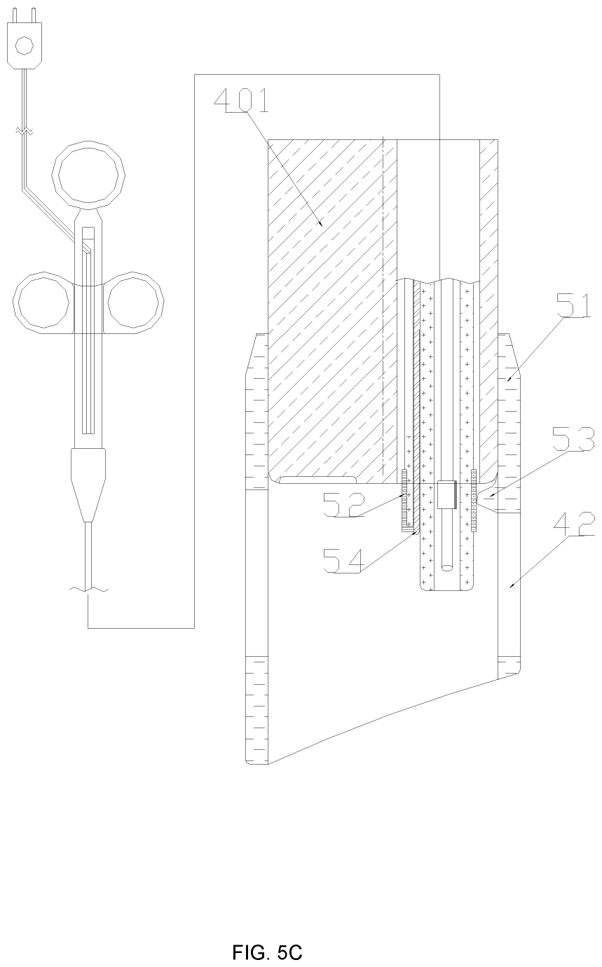

It should be noted that although the expandable frame 42 with a transparent cap 41 is provided in the foregoing embodiment, the foregoing two structures are not limited, provided that is an auxiliary apparatus for an endoscope can be tightly attached to a side wall of tissue and has a larger surface area than the sheath, so that the first electrically conductive part 51 may be attached to the peripheral surface of the peripheral surface of the expandable frame 42 and is connected to the second electrically conductive part 52 through the sliding contact part 53 to achieve the same effect. Preferably, as shown in FIG. 7A, the expandable frame 42 includes a sleeve 411′ and a flexible part or an elastic part 412 connected to the sleeve 411′, the sleeve 411′ is configured to be sleeved over the endoscope, and the sliding contact part 53 is fixed on an inner side of the expandable frame or an inner side of the sleeve 411′. The flexible part or elastic part 412 provides particular cushioning for the sliding contact part 53 or applies particular pressure to keep electrical contact between the sliding contact part 53 and the second electrically conductive part 52, so that the sliding contact part 53 is kept being configured to be electrically connected to the second electrically conductive part 52.

Further, as shown in FIG. 2 and FIG. 3, the second electrically conductive part 52 is fixed at a position, at a distance of L1 near a top end, on a periphery of a front section of the sheath 10, and is conductive. An outer diameter of the second electrically conductive part 52 does not exceed an inner diameter of the instrument passage 403. Moreover, referring to both FIG. 1B and FIG. 4, the second cavity 12 is formed in the sheath 10. A third electrically conductive part 54 having a conductive function is encapsulated in the second cavity 12. The second electrically conductive part 52 is electrically connected to the third electrically conductive part 54, and the second electrically conductive part 52 is electrically connected to a passive electrode 61 of the feeding electrode 60 through the third electrically conductive part 54, and an external power supply supplies power to the electric snare. The present invention is not limited thereto. Alternatively, a layer of a conductive material is provided on an outer surface of the sheath 10, and the conductive material forms the second electrically conductive part 52. The type of the conductive material may be selected as required. The layer of the conductive material at least covers a peripheral surface of a part of the sheath 10. The layer of the conductive material may be made very thin to reduce an increase in an additional volume. Preferably, the layer of the conductive material is made smooth to facilitate the movement of the sheath 10 in the instrument passage of the endoscope.

Although the third electrically conductive part 54 is encapsulated in the sheath 12 in this embodiment, a specific position of the third electrically conductive part 54 on the sheath is not limited, provided that the third electrically conductive part 54 is electrically isolated from the electric snare 221 in the first cavity 11 and is eventually electrically connected to the first electrically conductive part 51. FIG. 4 shows several arrangement manners of the third electrically conductive part 54 in the sheath 12.

Although the third electrically conductive part 54 has a linear shape in this embodiment, the specific shape and structure of the third electrically conductive part 54 are not limited, provided that the third electrically conductive part 54 can electrically connect the first electrically conductive part 51 to the feeding electrode and can bend in accordance with a flexible cavity.

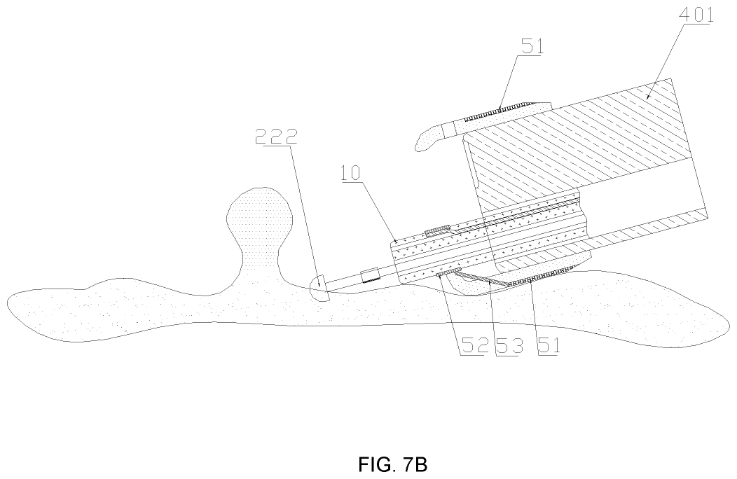

A working manner of the present invention is described below. Before operations of endoscopic surgery are started, the expandable frame 42 with the first electrically conductive part 51 is assembled at a distal end of an endoscope body 401 of the endoscope, and the sheath 10 with the electric snare 221 is further inserted in the instrument passage 403 of the endoscope. Next, the endoscope is inserted in a cavity (for example, a digestive tract, a vagina) of a human body, and an image transmitted by the camera 402 is simultaneously observed. When a focus is reached, a sliding ring 31 on the drive part 30 is operated, the electric snare 221 is closed around tissue to be excised, and the first electrically conductive part 51 is tightly attached to tissue 70 on a side wall at the same time. Finally, the feeding electrode 60 is turned on, and a current flows through the active electrode 62 of the feeding electrode 60, the electric snare 221, the tissue to be excised, and a return path 50 (as shown in FIG. 12, the return path 50 includes the first electrically conductive part 51, the sliding contact part 53, the second electrically conductive part 52, and the third electrically conductive part 54), and returns to the passive electrode 61 of the feeding electrode 60 to form a loop. High heat is generated at a contact position between the electric snare 221 and the tissue to be excised to excise the tissue. For the formation of the return path 50, and when the sheath 10 is located in a preset position, the sliding contact part 53 contacts and is electrically connected to the second electrically conductive part 52 and the sheath 10 is in sliding fit with the sliding contact part 53. The preset position is not necessarily a point, and it may be designed that when the sheath 10 slides within a particular range, the sliding contact part 53 can contact and be electrically connected to the second electrically conductive part 52. In this way, the first electrically conductive part 51, the sliding contact part 53, the second electrically conductive part 52, and the third electrically conductive part 54 form the return path 50 of the current guided out from the human body. In a conventional instrument for an endoscope, the return path 50 is not provided or no other manner is used to guide out a current, and a reliable electrical connection to the human body cannot be kept as well as in the present invention, easily resulting in accidental injury. Because the first electrically conductive part 51 is close to the electrical treatment part, a current that enters the human body is guided out by the first electrically conductive part 51 instead of flowing all over the human body. Therefore, a stronger current can be used, so as to obtain electrothermal efficiency that cannot be obtained by a conventional electric snare 221, and the stronger current generates more heat at the focus. The description of the active electrode 62 and the passive electrodes 61 herein and in the context is only used for ease of recognizing a current direction in a same loop, and an actual flowing direction of a current is not limited. A high-frequency alternating current with a frequency not less than 20 KHZ usually flows in the formed loop.

It should be noted that the foregoing electrical contact and electrical connection means that a current can be in practice conducted. For such contact, two entities may contact or the entities may not contact (there is a gap) but complete conduction of a current by means of conductive liquids (for example, tissue fluids, and secretions) in a human body.

The preferred solutions and beneficial effects of this embodiment are as follows:
1. The treatment apparatus for the endoscope includes: the first electrode, including the electrical treatment part and the operating wire 20, where the electrical treatment part is configured to perform an electrical operation on a human body, and the electrical operation includes, but is not limited to, an electric shock effect, an electric heating effect, and an electrical cauterization effect; the second electrode, configured to be installed inside the endoscope, and including the first electrically conductive part 51 and the sliding contact part 53 electrically connected to the first electrically conductive part 51, and the first electrically conductive part 51 being configured to contact a human body; and the sheath 10, the second electrically conductive part 52 being provided on the surface of the sheath 10, where the operating wire 20 is passed through the sheath 10, the sheath 10 is passed through the endoscope, the sheath 10 is movable back and forth in the instrument passage of the endoscope, and when the sheath 10 is located in a preset position, the sliding contact part 53 contacts and is electrically connected to the second electrically conductive part 52 and the sheath 10 is in sliding fit with the sliding contact part 53.

The first electrode performs an electrical operation on a human body, and a current that enters the human body is looped through the second electrically conductive part instead of flowing all over the human body, so as to avoid damage to other organs or electronic apparatuses (for example, a pacemaker) in the human body, thereby ensuring the safety of the entire operation. Provided that electrical requirements can be satisfied, the second electrode may be provided at any position in the endoscope, so that a sufficiently large area of contact between the second electrically conductive part 52 and the human body is ensured to fully guide out a current.
2. The treatment apparatus for the endoscope further includes the expandable frame 42. The expandable frame 42 is configured to be sleeved over the endoscope. The first electrically conductive part 51 is provided on a periphery of the expandable frame 42. A distance between a part, being configured to contact a human body, of the first electrically conductive part 51 and an axis of the endoscope is greater than a radius of the endoscope. The expandable frame 42 is used to increase the distance between the part, being configured to contact the human body, of the first electrically conductive part 51 and the axis of the endoscope. Because the sheath 10 is passed through the endoscope, the first electrically conductive part 51 contacts the human body first, thereby ensuring reliable contact between the first electrically conductive part 51 and the human body and avoiding electrical leakage.

Provided that electrical requirements can be satisfied, the expandable frame 42 may be provided at any position of the endoscope. Preferably, the expandable frame is provided at a distal end of the endoscope, and the expandable frame is kept as close as possible to the electrical treatment part of the first electrode, thereby reducing a path of a current flowing through the human body.
3. A conductive material is provided on the peripheral surface of the expandable frame 42, and the conductive material forms the first electrically conductive part 51. The type of the conductive material may be selected as required. The layer of the conductive material at least covers a peripheral surface of a part of the expandable frame 42. The layer of the conductive material may be made very thin to reduce an increase in an additional volume. Preferably, the layer of the conductive material is made smooth to reduce damage to the human body. In this embodiment, the expandable frame has a cylindrical shape, but is not limited thereto. Another shape may be used, provided that the expandable frame can be installed inside the endoscope.
4. The expandable frame 42 includes a sleeve and a transparent cover connected to the sleeve. The sleeve is configured to be sleeved over the endoscope. The transparent cover is transparent or translucent. The transparent cover is configured to allow the endoscope to obtain an image. When a part, blocking an optical device (including, but is not limited to, a camera, a lighting device) of the endoscope, of the cover of the expandable frame 42 is made transparent or translucent, it is ensured that the optical device acquires an image normally.
5. The expandable frame 42 includes a sleeve and a flexible part or an elastic part connected to the sleeve, the sleeve is configured to be sleeved over the endoscope, and the sliding contact part 53 is fixed on an inner side of the expandable frame, and is connected to the flexible part or elastic part; or the sliding contact part 53 may be fixed on an inner side of the sleeve. The flexible part or elastic part provides particular cushioning for the sliding contact part 53, so that the sliding contact part 53 is kept being electrically connected to the second electrically conductive part 52.
6. It may be selected as required that the electrical treatment part is the electric snare 221 or the electrocoagulation forceps 223 or the electric biopsy forceps 224 or a sphincterotome. The treatment apparatus for the endoscope of the present invention has a wide applicable range.
7. A layer of a conductive material is provided on an outer surface of the sheath 10, and the conductive material forms the second electrically conductive part 52. The type of the conductive material may be selected as required. The layer of the conductive material at least covers a peripheral surface of a part of the sheath 10. The layer of the conductive material may be made very thin to reduce an increase in an additional volume. Preferably, the layer of the conductive material is made smooth to facilitate the movement of the sheath 10 in the instrument passage of the endoscope.
8. The first cavity 11 and the second cavity 12 are provided on the sheath 10. The operating wire 20 is passed through the first cavity 11. The third electrically conductive part 54 is provided in the second cavity 12. The third electrically conductive part 54 is arranged in an axial direction along the sheath, and the third electrically conductive part 54 is electrically connected to the second electrically conductive part 52. The second cavity 12 is provided for special use by the third electrically conductive part 54, thereby ensuring that the third electrically conductive part 54 is electrically insulated from the second electrically conductive part 52 and the first electrode, thereby improving reliability.
9. The second cavity 12 has an annular shape surrounding the first cavity 11. The third conductive layer is distributed around the first cavity 11, so that an increase in the radius of the sheath 10 caused by the third electrically conductive part 54 may be restrained, so as to reduce the radius of the sheath 10.
10. The treatment apparatus for the endoscope further includes the feeding electrode 60. The feeding electrode 60 includes the active electrode 62 and the passive electrode 61. The electrical treatment part is electrically connected to the active electrode
62. The third electrically conductive part 54 is electrically connected to the passive electrode 61. The active electrode 62 supplies power to the electrical treatment part. The electrical treatment part performs an electrical operation on a human body. A current enters the human body from the electrical treatment part, is then guided out from the first electrically conductive part 51, and flows through the second electrically conductive part 52 to return to the passive electrode 61, so that the current flows by a minimum path in the human body.
11. The second electrically conductive part 52 surrounds the sheath 10 by one loop in a circumferential direction of the sheath 10, so that when the sheath 10 slides back and forth in the endoscope, desirable contact is kept between the second electrically conductive part 52 and the sliding contact part 53, and electrical contact is not affected even if the sheath 10 spins.

It should be noted that in this embodiment, the treatment apparatus for an endoscope may not include the second electrode. The second electrode may be externally connected to the treatment apparatus for an endoscope during use. In this case, the apparatus includes: a first electrode, including an electrical treatment part and an operating wire; and a sheath, provided on a surface thereof with a second electrically conductive part, where the second electrically conductive part is configured to be electrically connected to the first electrically conductive part on the endoscope. The arrangement of the remaining part of the sheath is the same as above. Details are not described herein again.

In this embodiment, the first electrically conductive part 51 is provided on the expandable frame 42 of the endoscope. An outer diameter of the expandable frame 42 is greater than an outer diameter of the endoscope body 401 of the endoscope, and the outer diameter of the endoscope body 401 of the endoscope is greater than an outer diameter of the sheath 10. Therefore, compared with the case in which the first electrically conductive part 51 is provided on the sheath 10, when the first electrically conductive part 51 is provided on the expandable frame 42, the first electrically conductive part 51 contacts a human body more easily to form an electrical loop, so as to ensure that “the electrical treatment part, the human body tissue, and the first electrically conductive part” form a stable current loop during surgery, thereby preventing the human body from burns.

Because the outer diameter of the expandable frame 42>the outer diameter of the endoscope body 401 of the endoscope>the outer diameter of the sheath 10, an area of contact between the first electrically conductive part 51 and the human body is large, a stronger surgical current may be used, and the surgery time is shortened. During surgery when a current is maintained within a safe current range, a length of safe contact of the first electrically conductive part 51 (the first electrically conductive part 51 provided inside the endoscope body 401) provided on the expandable frame 42 is far less than a length of the first electrically conductive part 41 provided on the sheath 10:

To facilitate description, for example, a diameter of a cutting head is 0.4 mm, an electrical cutting current is 680 mA, and an average surgery time is 2.1 s. According to the regulations in GB9706.4-2009 and IEC6060-2-2:2006, a safe threshold of a contact current for a human body is 1000 mA·s/cm².

To describe a relationship between an arrangement position of a contact electrode and a requirement of a safe contact length L, it is assumed that a common digestive endoscope with a relatively small size, that is, a duodenoscope, is chosen for measurement. An outer diameter of the duodenoscope is d₁=1.25 cm, and a maximum outer diameter of the treatment apparatus does not exceed an inner diameter of an endoscope channel d₂=0.32 cm.

(1) The first electrically conductive part 51 is provided on the sheath 10.

It is assumed that when the first electrically conductive part 51 is provided on the sheath 10 (that is, the first electrically conductive part 51 is provided on the peripheral surface of the sheath 10 and has an annular shape), an area of contact between the first electrically conductive part 51 and a human body is:

$${S}_{1} = \frac{90 °}{360 °} \cdot {d}_{2} \cdot L \cdot π ,$$

where

$$\frac{90 °}{360 °}$$

means that assuming that the first electrically conductive part 51 has an annular shape surrounding the peripheral surface of the sheath 10 by one loop, a range of contact between the first electrically conductive part 51 and the human body tissue is ¼ of the arc surface (that is, only 90° of the circumferential 360° contacts human body tissue).

To satisfy the safe threshold of the contact current for the human body:

$$\frac{680 mA \cdot 2.1 s}{{S}_{1}} < 1000 mA \cdot s / {cm}^{2} = > \frac{1428 mA \cdot s}{\frac{1}{4} \cdot 0.32 cm \cdot π \cdot {L}_{1}} < 1000 mA \cdot s / {cm}^{2} = > {L}_{1} < \frac{1428 cm}{80 π} = 5.68 cm .$$

Therefore, assuming that the first electrically conductive part 51 is provided on the sheath 10, to satisfy the safe threshold of the contact current for the human body, a length of the first electrically conductive body 51 in an axial direction of the sheath needs to be greater than 5.68 cm, or otherwise a deep burn may occur in the human body during normal surgery.

(2) The first electrically conductive part 51 is provided inside the endoscope body 401.

Assuming that the first electrically conductive part 51 is provided inside the endoscope body 401, a maximum area of contact with human body tissue is:

$${S}_{2} = \frac{90 °}{360 °} \cdot {d}_{1} \cdot L \cdot π .$$

To satisfy the safe threshold of the contact current for the human body:

$$\frac{680 mA \cdot 2.1 s}{{S}_{2}} < 1000 mA \cdot s / {cm}^{2} = > \frac{1428 mA \cdot s}{\frac{1}{4} \cdot 1.25 cm \cdot π \cdot {L}_{1}} < 1000 mA \cdot s / {cm}^{2} = > {L}_{2} < \frac{1428 cm}{312.5 π} = 1.45 cm .$$

As may be seen from above, the required length of the first electrically conductive part in the safe range is L₁»L₂. When the first electrically conductive part is provided on the peripheral surface of the expandable frame, the safe contact length required to prevent a deep burn is shorter than the length of the first electrically conductive part provided on the peripheral surface of the sheath by approximately 75%, so that the safe contact length can be reached more easily for the contact electrode during surgery, and the surgery is safer and more effective.

### Embodiment 2

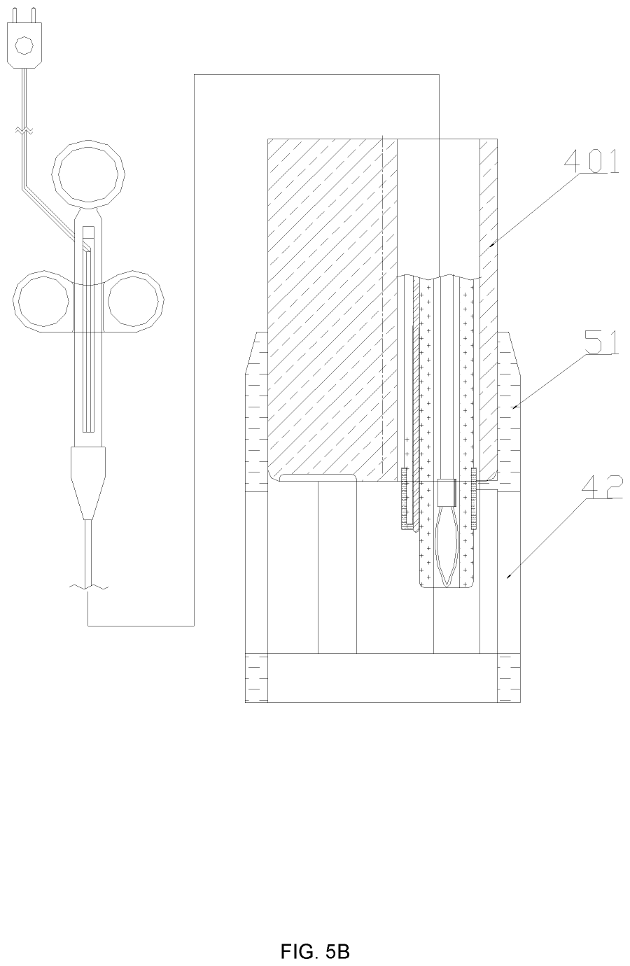

Embodiment 2 protects an endoscope. FIG. 6A, FIG. 6B, and FIG. 6C show three manners of guiding out a current via an endoscope. A fourth electrically conductive part 55 is provided inside the endoscope, and the fourth electrically conductive part 55 is electrically connected to the first electrically conductive part 51.

In a first implementation, the fourth electrically conductive part 55 is provided on an inner wall of the instrument passage 403 of the endoscope. The first electrically conductive part 51 is provided on a periphery of the camera 402 at the distal end of the endoscope body 401, and is electrically connected to the fourth electrically conductive part 55 in the instrument passage 403 directly or through the sliding contact part 53.

In a second implementation, the fourth electrically conductive part 55 is provided on an outer wall of the endoscope body 401 of the endoscope, and the fourth electrically conductive part 55 and the first electrically conductive part 51 are integrally provided on a periphery of the endoscope body 401 to form a whole.

In a third implementation, the fourth electrically conductive part 55 is provided on the inner wall of the instrument passage 403 of the endoscope. The first electrically conductive part 51 is provided on the periphery of the camera 402 at the distal end of the endoscope body 401, and the sliding contact part 53 passes through a side wall of the endoscope body 401 to electrically connect the first electrically conductive part 51 to the fourth electrically conductive part 55 in the instrument passage 403.

Preferred solutions and beneficial effects of this embodiment are as follows:
1. The endoscope includes the first electrically conductive part 51 provided on the outer surface of the endoscope. The first electrically conductive part 51 is configured to contact a human body. An instrument passage is provided in the endoscope. The instrument passage is configured to be passed through by a treatment apparatus for an endoscope. The fourth electrically conductive part 55 is provided inside the endoscope. The first electrically conductive part 51 is electrically connected to the fourth electrically conductive part 55. The first electrically conductive part 51 and the fourth electrically conductive part 55 provide the endoscope with an electrical path for guiding out a current in a human body. The current that enters the human body is guided out by the first electrically conductive part 51 and then guided out by the fourth electrically conductive part 55 in the endoscope.

Preferably, the second electrically conductive part 52 is configured to be electrically connected to the passive electrode 61 of the feeding electrode 60.
2. The first electrically conductive part 51 is directly provided inside the endoscope, the first electrically conductive part 51 may be embedded in an outer wall of the endoscope, or a layer of a conductive material may be provided on the peripheral surface of the endoscope, and the conductive material forms the fourth electrically conductive part 55.
3. A connecting through hole is formed in the endoscope. The peripheral surface of the endoscope and the instrument passage are communicated through the connecting through hole. The conductive material is provided in the connecting through hole to electrically connect to the first electrically conductive part 51 to the fourth electrically conductive part 55. After being guided out from the first electrically conductive part 51, the current that enters the human body sequentially passes through the conductive material in the connecting through hole and the fourth electrically conductive part 55 before being guided out.
4. The conductive material is provided on an end surface at the distal end of the endoscope to electrically connect the first electrically conductive part 51 to the fourth electrically conductive part 55. With such an arrangement, the end surface of the endoscope is also a position for contact with the human body. The end surface and the peripheral surface of the endoscope may both guide out a current from the human body when contacting the human body, so that an area of electrical contact with the human body may be enlarged.

### Embodiment 3

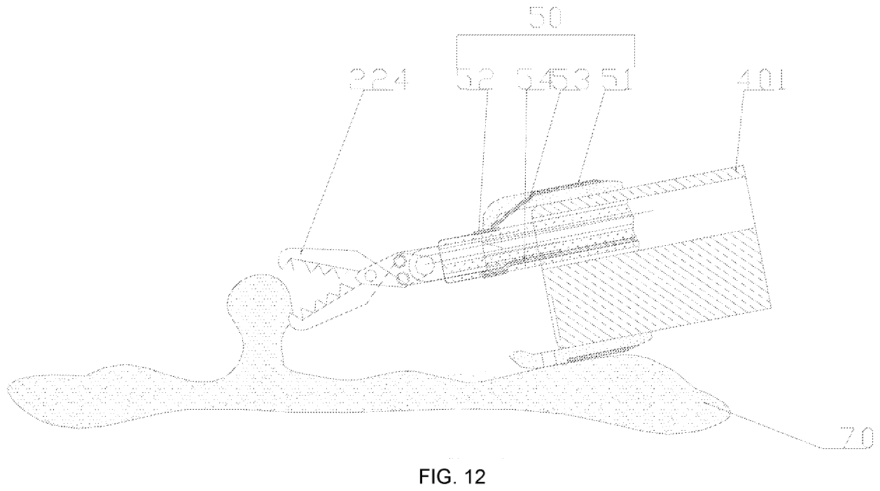

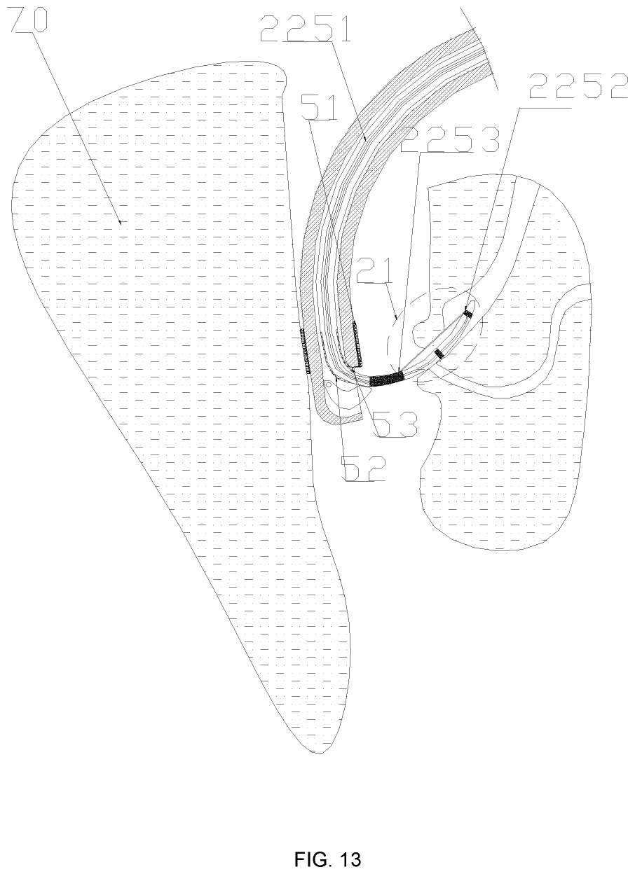

Embodiment 3 protects an expandable frame for another endoscope. As shown in FIG. 22 and FIG. 23, a conductive material is provided on the peripheral surface of the expandable frame 42, and the conductive material forms the first electrically conductive part 51. A proximal end of the first electrically conductive part 51 is electrically connected to the fourth electrically conductive part 55. The fourth electrically conductive part 55 (which may be a conductor wire) is arranged along the endoscope body 401, is guided out from a proximal end of the endoscope body 401, and is electrically connected to the passive electrode 61. During working, the expandable frame 42 is installed on an ordinary endoscope, so that the expandable frame 42 is tightly attached to human body tissue. An existing monopolar instrument is inserted in the instrument passage 403 at the same time. A power supply is turned on after a focus is reached, and a working circuit is formed among an electrical treatment part of the monopolar instrument, tissue to be treated, and the first electrically conductive part 51, so as to complete surgical operations.

The differences between Embodiment 3 and Embodiment 1 lie in that the fourth conductive body instead of the sliding contact part is provided on the expandable frame, and the fourth conductive body is passed through the endoscope or provided outside the endoscope. Preferably, the fourth conductive body is a conductor wire, and the conductor wire is passed through the endoscope or provided outside the endoscope. The remaining shape of the expandable frame may be consistent with that in Embodiment 1. Such an expandable frame is conveniently used. The first electrically conductive part of the expandable frame is provided on a periphery of the expandable frame and contacts a human body and guides out a current in the human body. Next, the fourth electrically conductive part guides out the current in the human body, so as to provide a passage for guiding out the current from the human body. The fourth electrically conductive part may be easily installed inside the endoscope in a sleeving manner, it is not necessary to make additional changes to the endoscope, thereby achieving high compatibility and a wide use range.

### Other Variants

The technical solutions in the foregoing embodiments may be implemented separately or implemented in combination, but is not limited to the foregoing embodiments. For example, the following variants may be used.

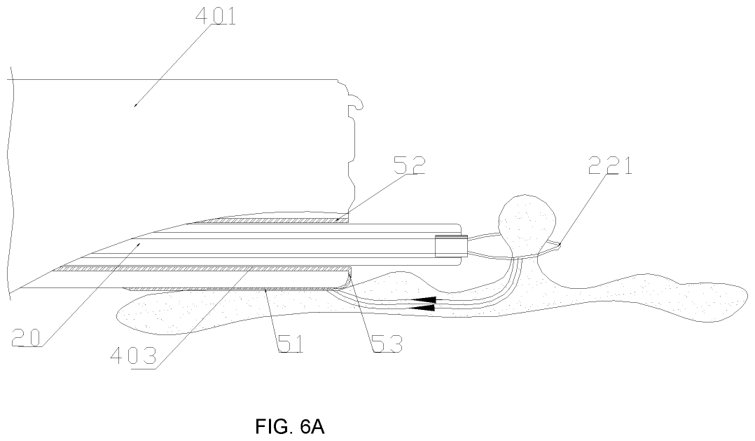

For example, to perform ESD surgery, as shown in FIG. 7A and FIG. 7B, the electric snare 221 is replaced with an ESD knife 222 having a head part extending in an axial direction of the operating wire. The head part 2221 may be a needle knife or may be a T-shaped knife, a star-shaped knife, provided that the head part is applicable to ESD surgery. FIG. 8A to FIG. 8F show several shapes of the electrical treatment part. The structural parts other than the ESD knife 222 are the same as those in the foregoing embodiments, and the details of these parts are omitted herein.

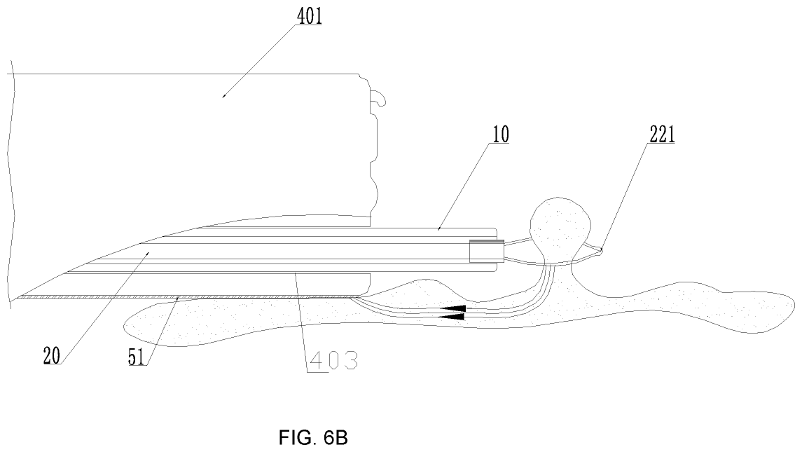

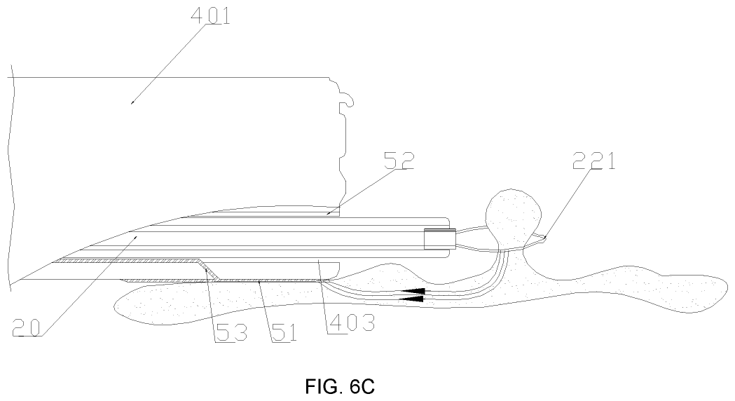

For another example, to perform hemostasis during surgery, as shown in FIG. 9 and FIG. 10, the electric snare 221 is replaced with the electrocoagulation forceps 223 having a pair of holding parts that can be opened or closed and can incise living tissue. The structural parts other than the electrocoagulation forceps 223 are the same as those in the foregoing embodiments, and the details of these parts are omitted herein.

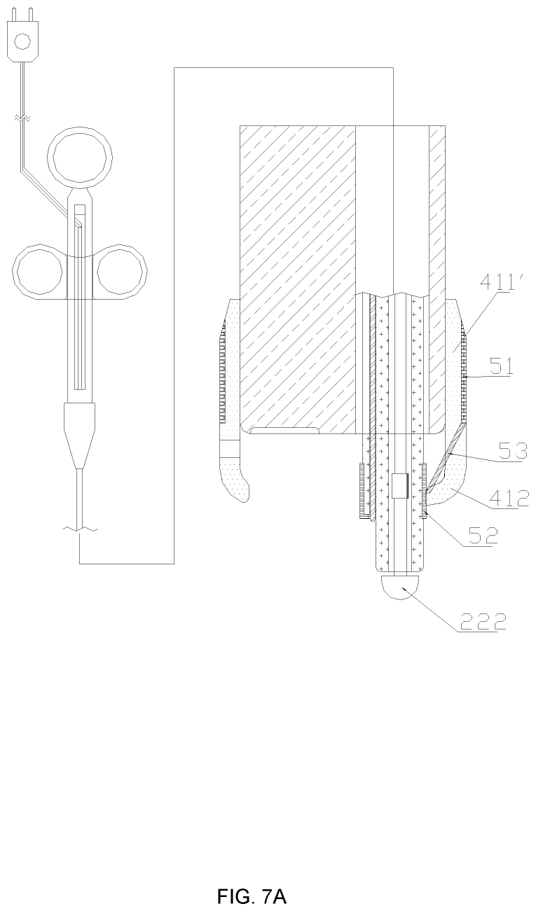

For still another example, to perform biopsy and hemostasis, as shown in FIG. 11 and FIG. 12, the electric snare 221 is replaced with the electric biopsy forceps 224. The structural parts other than the electric biopsy forceps 224 are the same as those in the foregoing embodiments, and the details of these parts are omitted herein.

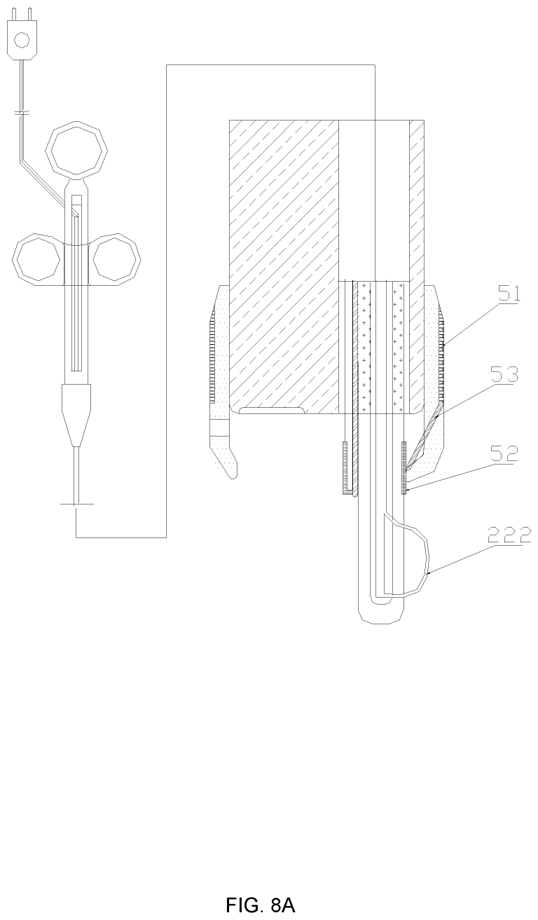

For yet another example, to perform endoscopic retrograde cholangiopancreatography (ERCP), as shown in FIG. 13, the electric snare 221 is replaced with a cutting wire 2252 having a papilla sphincterotome with a function of incising papillary sphincters. The papilla sphincterotome includes a conductor cavity 2251 extending longitudinally in the sheath 10 along the sheath 10. The conductor cavity 2251 is equivalent to the first cavity 11 in the embodiment of the electric snare 221. The cutting wire 2252 is accommodated in the conductor cavity. The cutting wire 2252 is passed through an anchor 2253 at a distance L from a distal end of the sheath and is joined to the distal end of the sheath, so that the cutting wire 2252 and the distal end form a bow-shaped cutting part 21. The cutting wire 2252 is securely joined to the sheath 10 at the anchor. A proximal end of the cutting wire 2252 is electrically connected to the operating wire 20 having conductive performance. The remaining structural parts in this embodiment are the same as those in the foregoing embodiments, and the details of these parts are omitted herein.

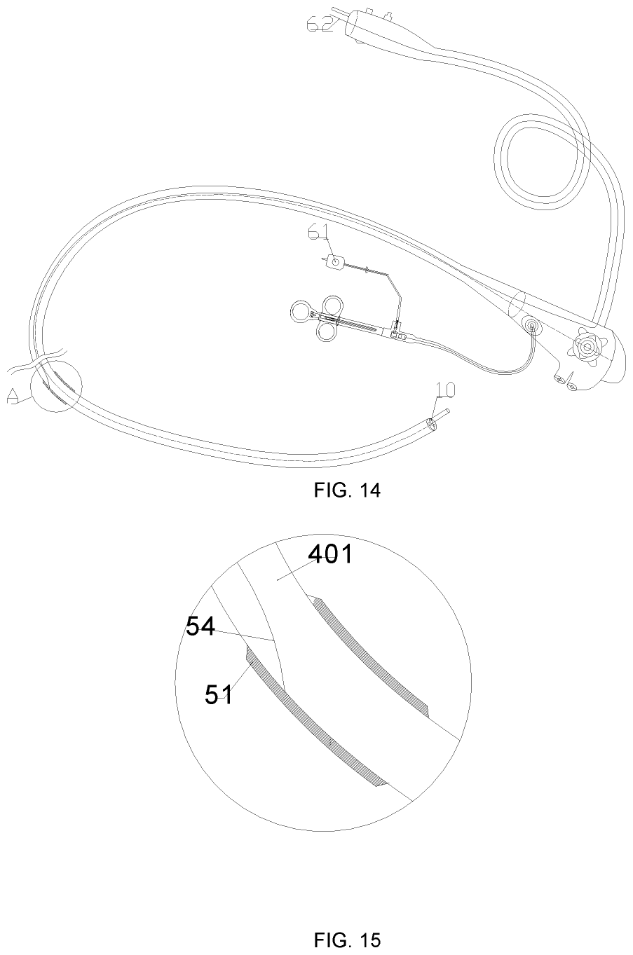

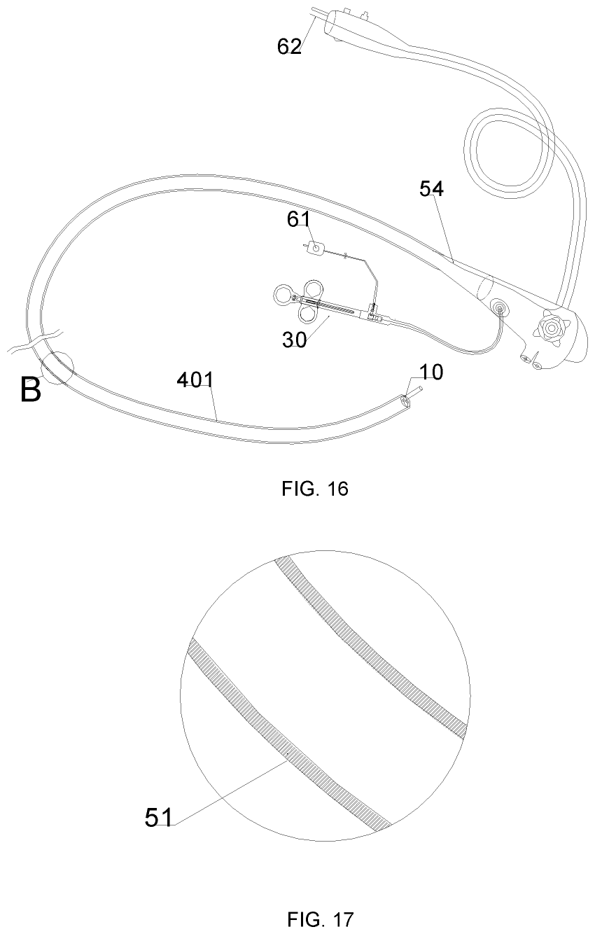

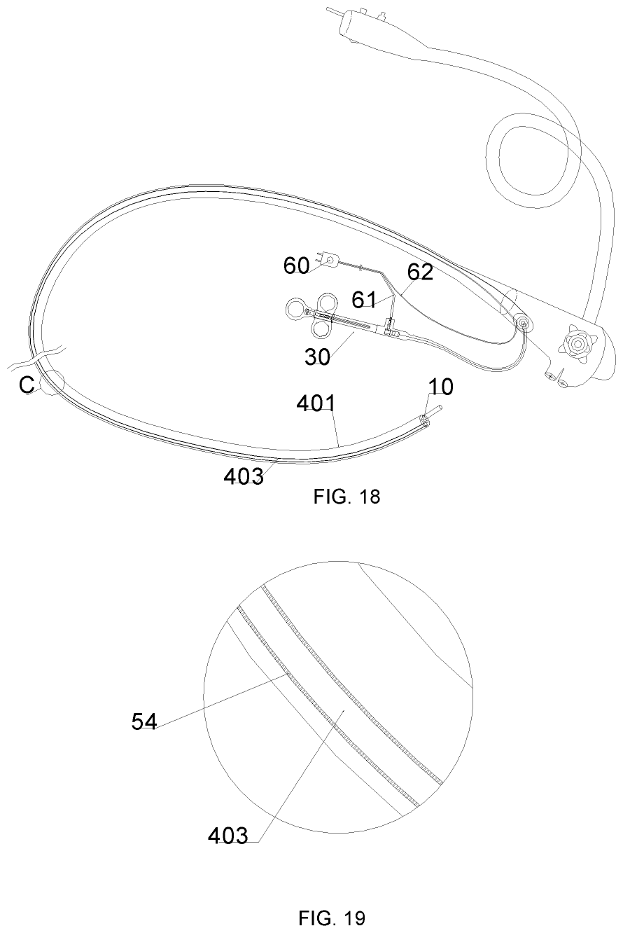

In addition, preferably, as shown in FIG. 24 to FIG. 26, the sliding contact part 53 may further include a fifth electrically conductive part 531 and a sixth electrically conductive part 532. The fifth electrically conductive part 531 may ensure a stable connection between the sliding contact part and the second electrode. The sixth electrically conductive part 532 is configured to be electrically connected to the first electrically conductive part 51. In FIG. 24 to FIG. 26, the fifth electrically conductive part is a steel spring ball. A highest point of the steel spring ball protrudes from an extension line of the instrument passage of the endoscope, and a tail of the steel spring ball is inserted in the expandable frame. The fifth electrically conductive part is electrically connected to the first electrically conductive part 51 through the sixth electrically conductive part. In addition, there may be another implementation of the fifth electrically conductive part 531. For example, the fifth electrically conductive part 531 may be provided as an electrically conductive ring. A central line of the electrically conductive ring is consistent with a central axis of the instrument passage of the endoscope, and an inner diameter of the electrically conductive ring is consistent with an inner diameter of the instrument passage. In this case, the sixth electrically conductive part 532 is electrically connected to the first electrically conductive part 51 and the electrically conductive ring. The electrically conductive ring is used to keep stable contact of the electrically conductive parts in all directions and further reduce manufacturing costs. The fifth electrically conductive part may also be an electrically conductive prism. A central line of the electrically conductive prism is consistent with a longitudinal axis of the endoscope, and the electrically conductive prism protrudes from an axial extension line of a wall of the instrument passage of the endoscope. Alternatively, the fifth electrically conductive part may be an insertion tube inserted at a distal end of the instrument passage, and may ensure stable matching between the sliding contact part and the instrument passage.

In addition, the first electrically conductive part 51 may also be arranged in another manner.

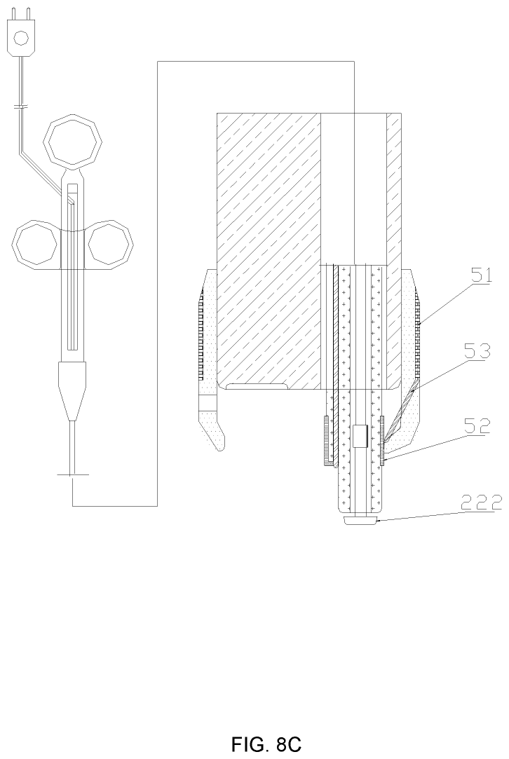

In a variant, as shown in FIG. 14 and FIG. 15, the first electrically conductive part 51 is provided in the middle of the endoscope body 401 of the endoscope. The return conductor 54 is provided longitudinally along the endoscope body 401, and is electrically connected to the first electrically conductive part 51. The feeding power supply 60 includes the passive electrode 61 and the active electrode 62. The passive electrode 61 is electrically connected to the third electrically conductive part 54 (or the fourth electrically conductive part 55), and is guided out at the proximal end of the endoscope body 401. In addition, the active electrode 62 is electrically connected to a high-frequency treatment part, and is guided out from the drive part 30. Although the third electrically conductive part 54 (or the fourth electrically conductive part 55) is provided inside the endoscope body 401 in this embodiment, a specific position of arranging the third electrically conductive part 54 is not limited, provided that the third electrically conductive part 54 (or the fourth electrically conductive part 55) can be electrically connected to the first electrically conductive part. The third electrically conductive part 54 (or the fourth electrically conductive part 55) may be arranged on the sheath 10, and is electrically connected to the first electrically conductive part 51 through a conduction mechanism provided on an inner side of the first electrically conductive part 51. In this case, the passive electrode 61 and the active electrode 62 are both guided out from the drive part 30.

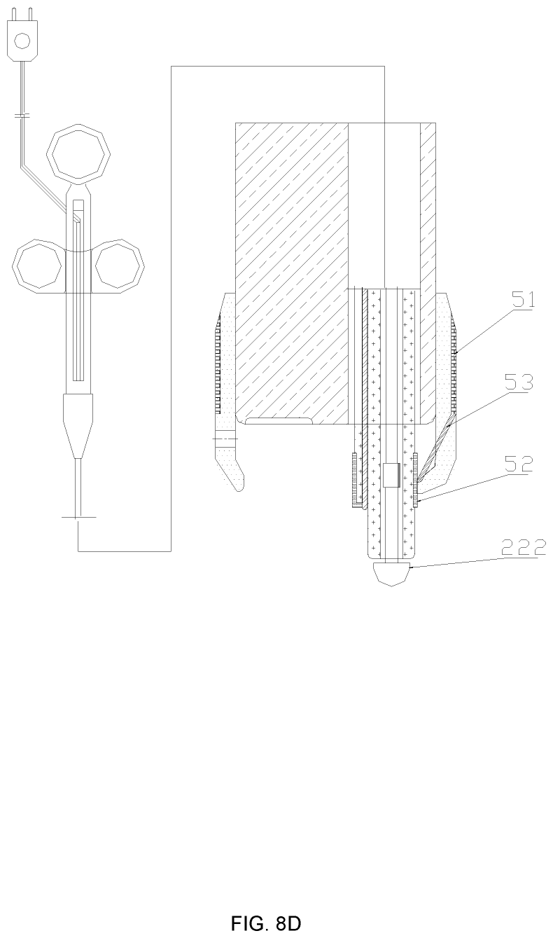

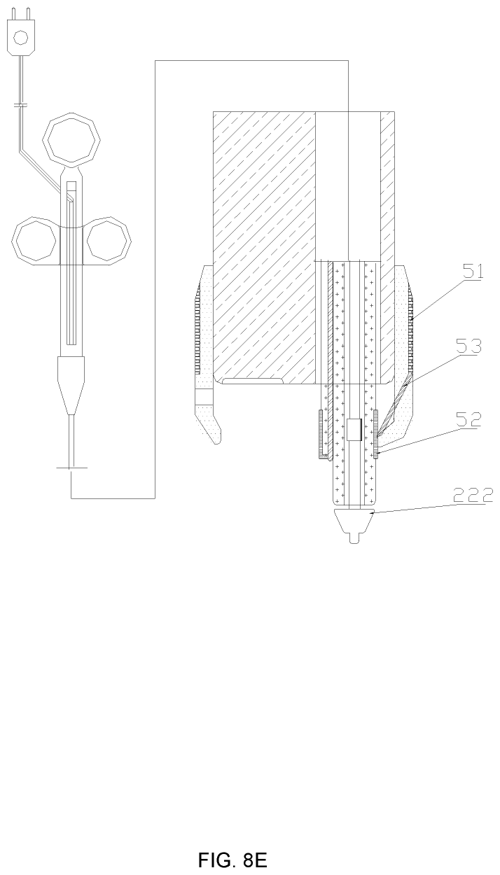

In another variant example, FIG. 16, FIG. 17, and FIG. 6B show an endoscope. A first electrically conductive part 51 of the endoscope covers an outer surface of the endoscope body 401. The return conductor 54 is electrically connected to the proximal end of the first electrically conductive part 51, and is guided out from the proximal end of the endoscope body 401 to the passive electrode 62. The active electrode 61 is electrically connected to the high-frequency treatment part, and is guided out from the drive part 30.

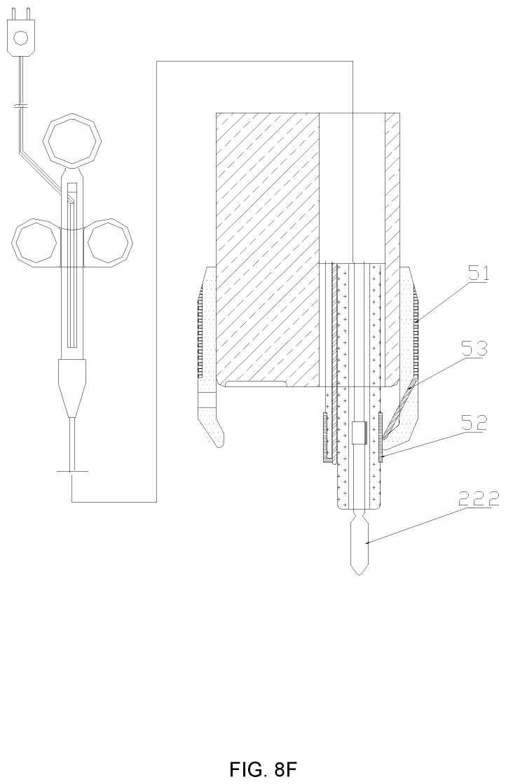

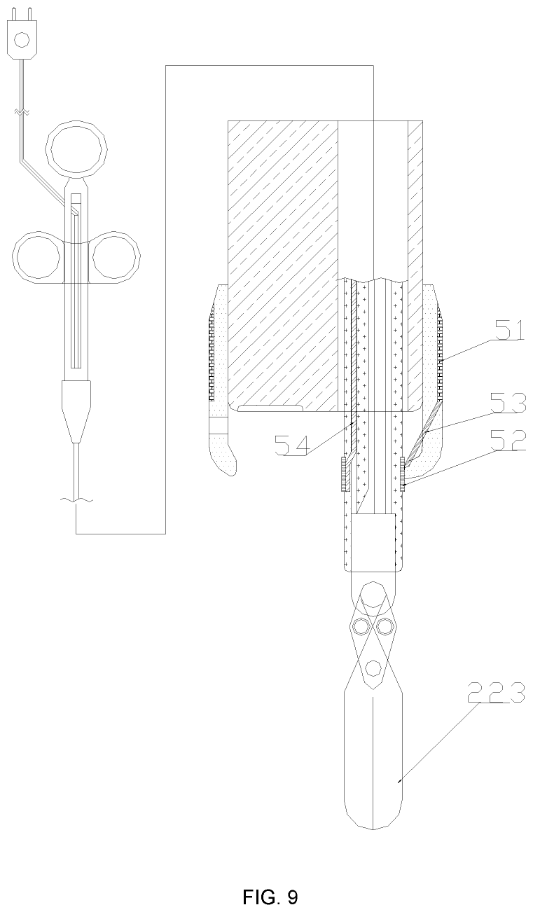

In another variant example, FIG. 18, FIG. 19, and FIG. 6A show another endoscope. The first electrically conductive part 51 of endoscope covers a peripheral surface at the distal end of the camera 401. The return conductor 54 is arranged on an inner wall of the instrument passage 403 and is electrically connected to the passive electrode 61, and the return conductor 54 and the active electrode 62 are then guided out together from the drive part 30.

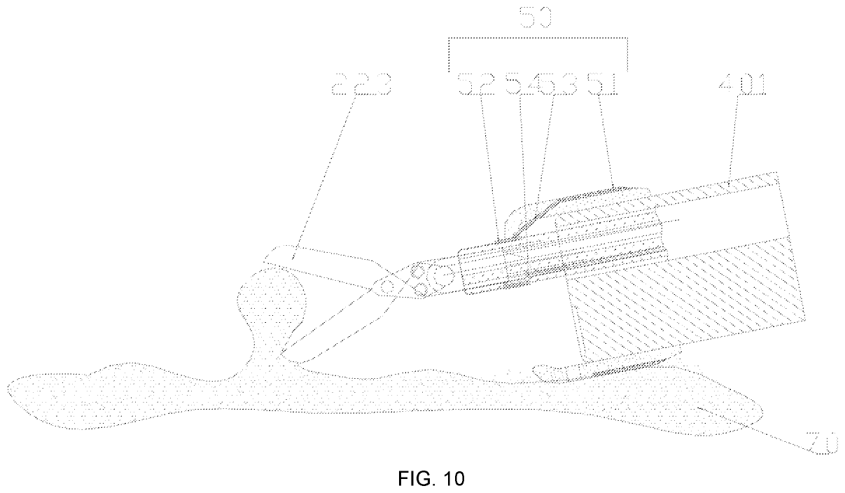

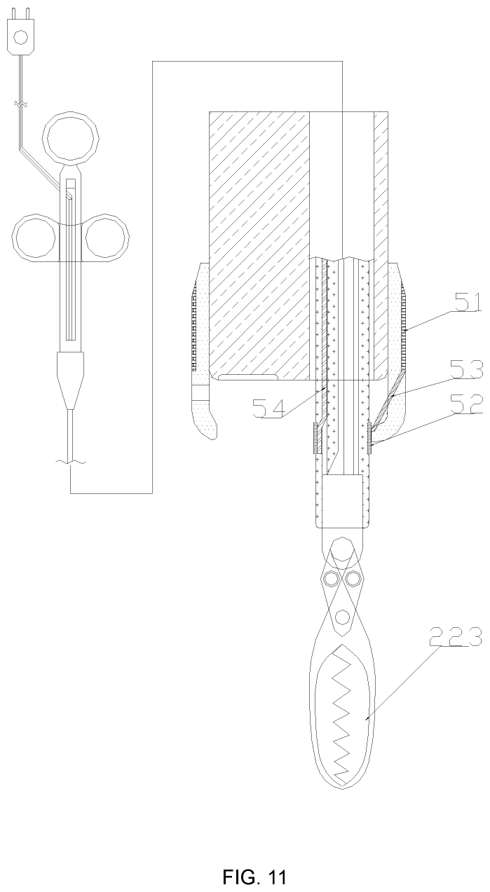

In another variant example, FIG. 20 and FIG. 21 show another endoscope. The return conductor 54 of the endoscope is a conductor wire arranged on an outer side of the endoscope body 401, and during use, the return conductor 54 coordinate with a high-frequency treatment apparatus having the first electrically conductive part 51. The conductor wire is electrically connected to the first electrically conductive part 51.

### Embodiment 4

Differences between Embodiment 4 and Embodiment 1 are as follows:

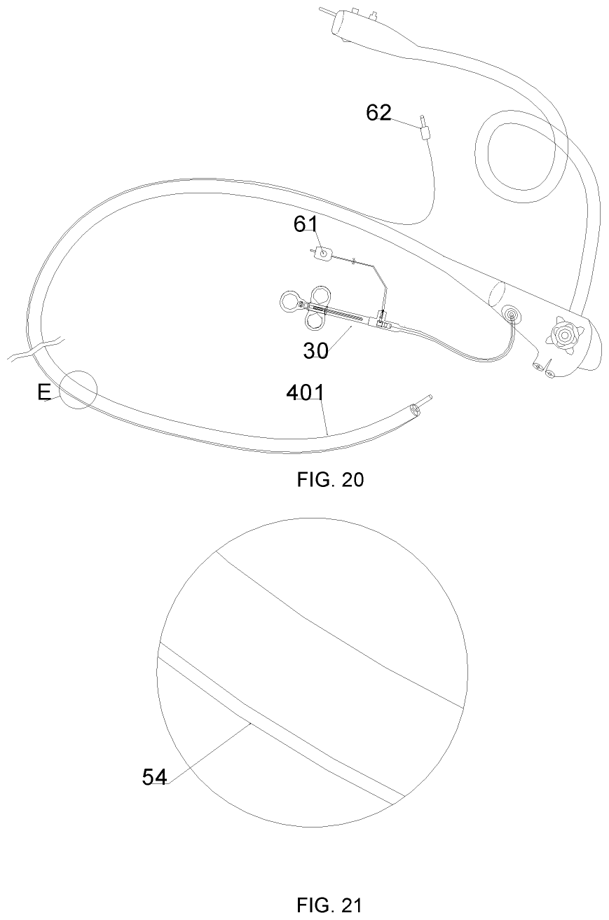

As shown in FIG. 27 to FIG. 30, the application structure for an endoscope further includes a tissue operation mechanism 70. The tissue operation mechanism 70 is installed on the expandable frame 42. The expandable frame 42 is sleeved over the endoscope body 401 of the endoscope 40. A mechanism installing hole 411 is further formed in the expandable frame. The tissue operation mechanism 70 is passed through the mechanism installing hole 411. There are two first electrically conductive parts 51. One first electrically conductive part 51 is provided on the tissue operation mechanism, and the other first electrically conductive part 51 is provided on the peripheral surface of the expandable frame. The first electrically conductive part 51 of the tissue operation mechanism 70 is electrically connected to the first electrically conductive part 51 of the expandable frame 42. The first electrically conductive part 51 provided on the tissue operation mechanism 70 may be directly provided on an outer surface of the tissue operation mechanism 70, or an independent mechanism may be provided as the first electrically conductive part 51 on the tissue operation mechanism 70, provided that the first electrically conductive part 51 can be electrically connected to human body tissue. Specific forms are not limited.

The fourth electrically conductive part 55 is further provided on the tissue operation mechanism 70. The first electrically conductive part 51 and the fourth electrically conductive part 55 that are provided on the tissue operation mechanism 70 are electrically connected.

The first electrically conductive part 51 is configured to contact a human body to form an electrical loop. One or both of the first electrically conductive part 51 on the expandable frame 42 and the first electrically conductive part 51 of the tissue operation mechanism 70 electrically contacts human body tissue, so as to guide out a current from the human body to form the loop. Two first electrically conductive parts 51 are provided to make it easier to electrically contact human body tissue, thereby ensuring the security of surgery and avoiding burning of human body tissue.

As shown in FIG. 27, the tissue operation mechanism 70 is installed outside the endoscope, and the tissue operation mechanism 70 and the electrical treatment part (the electrocoagulation forceps 223) are connected to the same high-frequency power supply.

The tissue operation mechanism 70 is an apparatus from performing operations on human body tissue. Any type of tissue operation mechanism 70 may be selected according to operations required in surgery, and the operations include, but are not limited to, holding tissue, snaring tissue, pushing tissue to create a space, pulling tissue, and fixing the position of tissue. Because a surgical procedure usually includes several operations, and an apparatus that can complete an operation other than the function of the treatment apparatus for an endoscope in the present invention may be regarded as a tissue operation apparatus in the present invention.

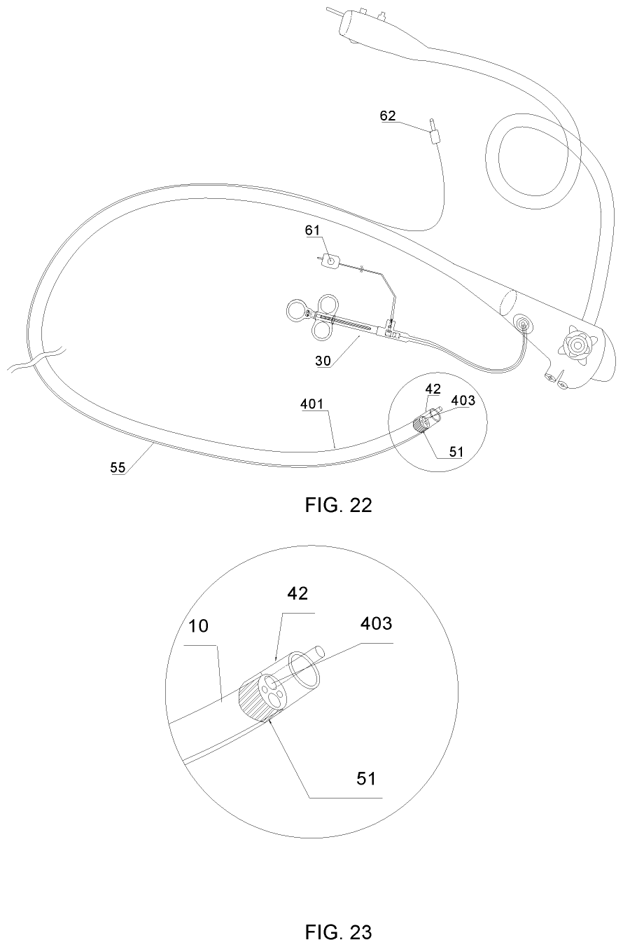

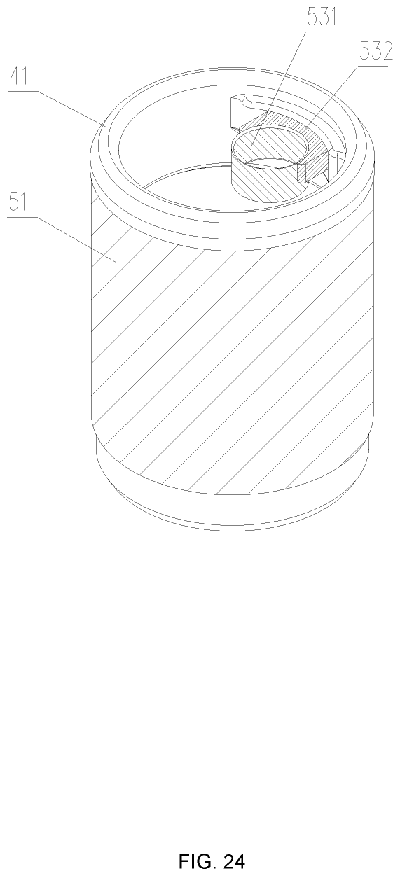

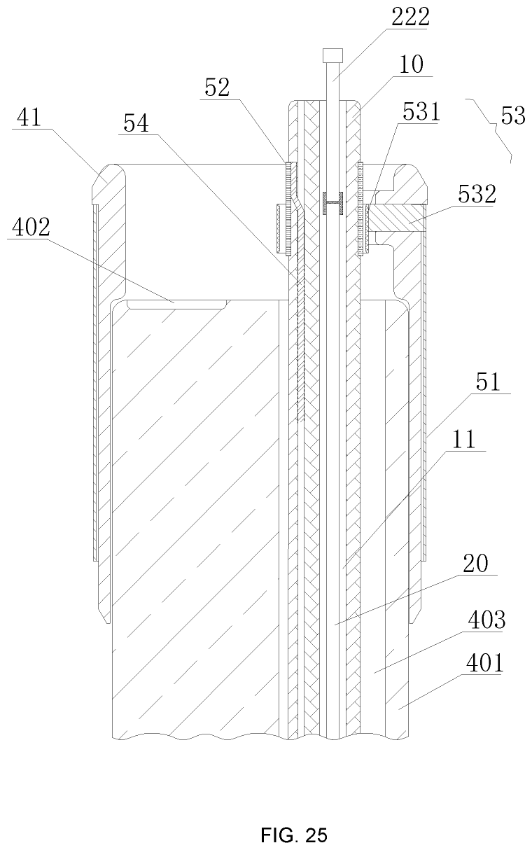

A method of using the treatment apparatus for an endoscope is as follows:
(1). FIG. 27 and FIG. 28 show a state before the treatment apparatus enters a human body cavity. The treatment apparatus for an endoscope is accommodated in the instrument passage 403 of the endoscope body 401. The tissue operation mechanism 70 does not extend beyond a front end of the endoscope body 401. A holding mechanism is provided at a front end of the tissue operation mechanism 70. The holding mechanism is conductive. The holding mechanism at the front end of the tissue operation mechanism 70 forms the first conductor part 51. A conductive wire is provided in the tissue operation mechanism 70 or the tissue operation mechanism 70 is conductive. The conductive wire or the tissue operation mechanism 70 forms the fourth conductor part 55 (FIG. 28 shows a case in which the conductive wire is provided in the tissue operation mechanism 70).
(2). The position of a focus is reached. The electrocoagulation forceps 223 and the tissue operation mechanism 70 of the treatment apparatus for an endoscope are deployed. As shown in FIG. 29, the first electrically conductive part 51 first electrically contacts a human body, and the electrocoagulation forceps 223 then contacts the human body. When the electrocoagulation forceps 223 is used to operate on the human body, there is usually hindrance or blockage of human body tissue or human body tissue beats with breathing pulses. Therefore, the tissue operation mechanism 70 is first used to operate on the human body tissue (push the tissue to create a space, fix the tissue or the like). FIG. 30 shows that the tissue operation mechanism 70 pushes tissue to create a space. While the tissue operation mechanism 70 is used to operate on the human body tissue, the first electrically conductive part 51 naturally contacts the human body. That is, the first electrically conductive part 51 electrically contacts the human body. The electrocoagulation forceps 223 is then operated to operate on the human body. At this time, the electrocoagulation forceps 223 contacts the human body, and the treatment apparatus for an endoscope, the human body tissue, the tissue operation apparatus, and the power supply form an electrical loop.

In this case, as shown in FIG. 30, the treatment apparatus for an endoscope is a monopolar apparatus, and the tissue operation apparatus is a monopolar apparatus. The first electrically conductive part is configured to contact a human body to form an electrical loop. The fourth electrically conductive part is grounded or connected to a passive power supply. The treatment apparatus for an endoscope, the human body tissue, the tissue operation apparatus, and the power supply form an electrical loop.

### Embodiment 5

Differences between Embodiment 5 and Embodiment 1 are as follows:

The expandable frame 42 is silicone rubber that contains several conductive particles, and the conductive particles form the first electrically conductive part. That is, the expandable frame 42 is conductive. The expandable frame forms the first electrically conductive part 51 and the sliding contact part 53, and an additional first electrically conductive part 51 and sliding contact part 53 do not need to be provided.

### Embodiment 6

Differences between Embodiment 6 and Embodiment 2 are as follows:

The expandable frame 42 is silicone rubber that contains several conductive particles, and the conductive particles form the first electrically conductive part. That is, the expandable frame 42 is conductive. The expandable frame forms the first electrically conductive part 51 and the sliding contact part 53, and an additional first electrically conductive part 51 and sliding contact part 53 do not need to be provided.

### Embodiment 7

Differences between Embodiment 7 and Embodiment 3 are as follows:

The expandable frame 42 is silicone rubber that contains several conductive particles, and the conductive particles form the first electrically conductive part. The expandable frame 42 is conductive. The expandable frame forms the first electrically conductive part 51, and an additional first electrically conductive part does not need to be provided.

In the present invention, a space of contact between an outer side of the endoscope body of the endoscope and tissue is fully used to increase a conductive area of a contact part between the treatment apparatus for an endoscope and tissue in a return path, so that while the risk of bipolar instrument burns is reduced, a thermal effect between the high-frequency treatment part and tissue to be excised and electrocoagulated is further improved, thereby improving the security and operation efficiency of endoscopic surgery, and ensuring further clinical popularization and application of bipolar instruments.

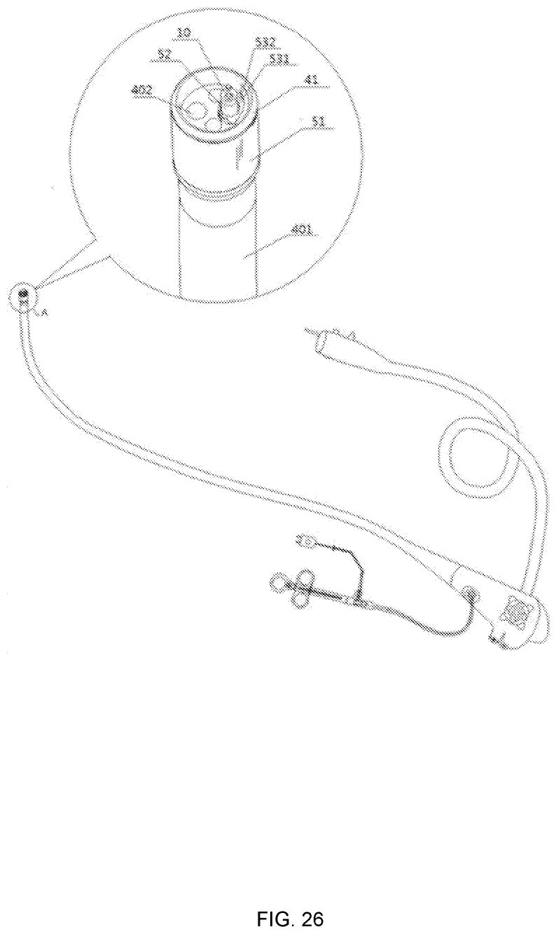

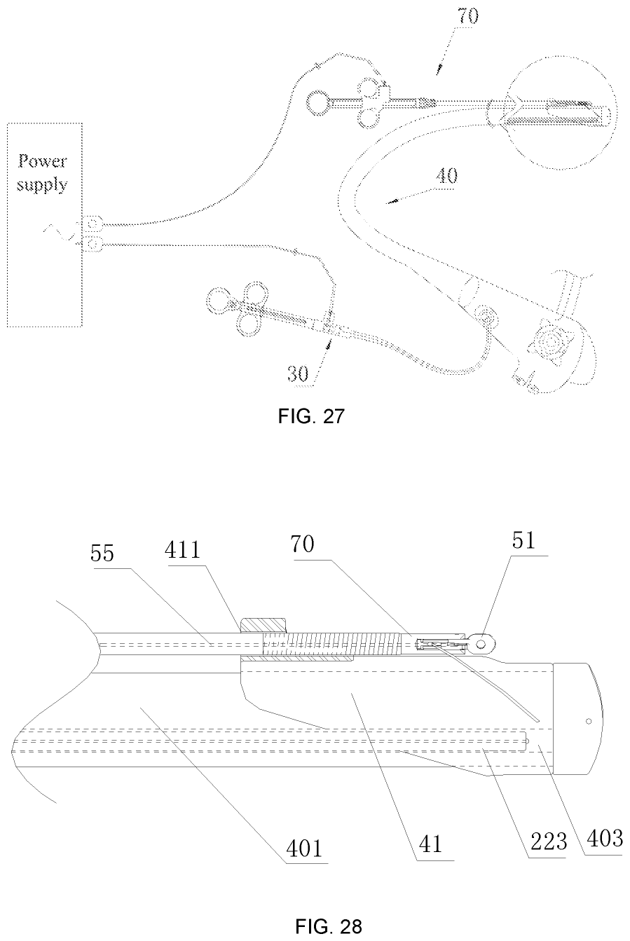

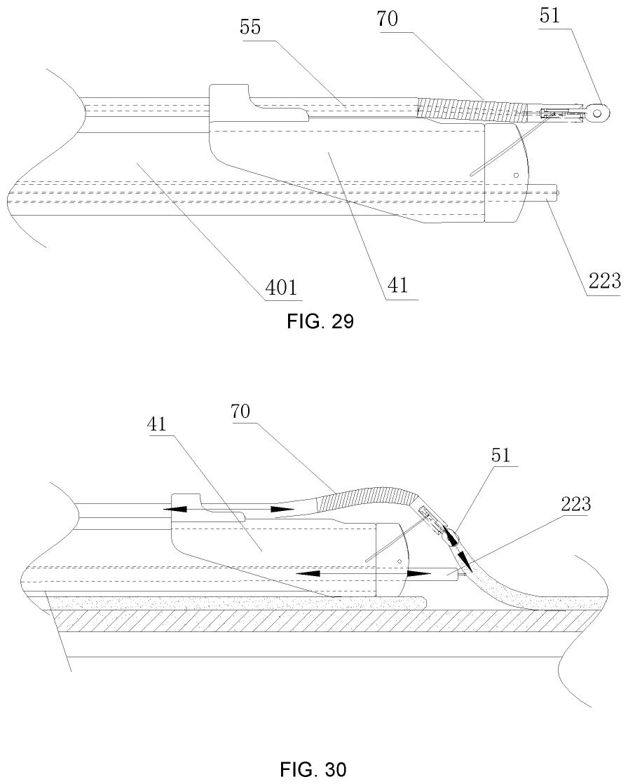

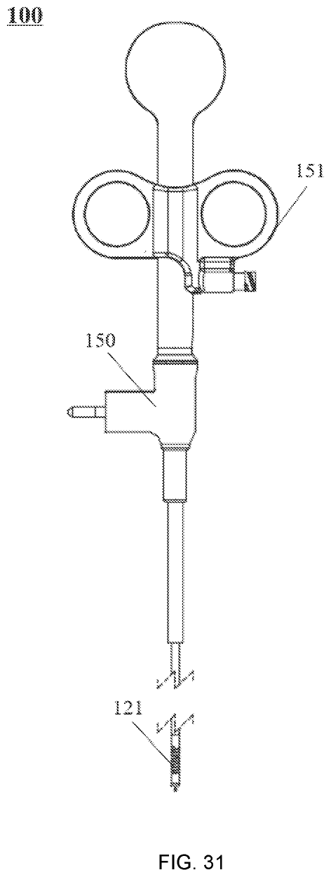

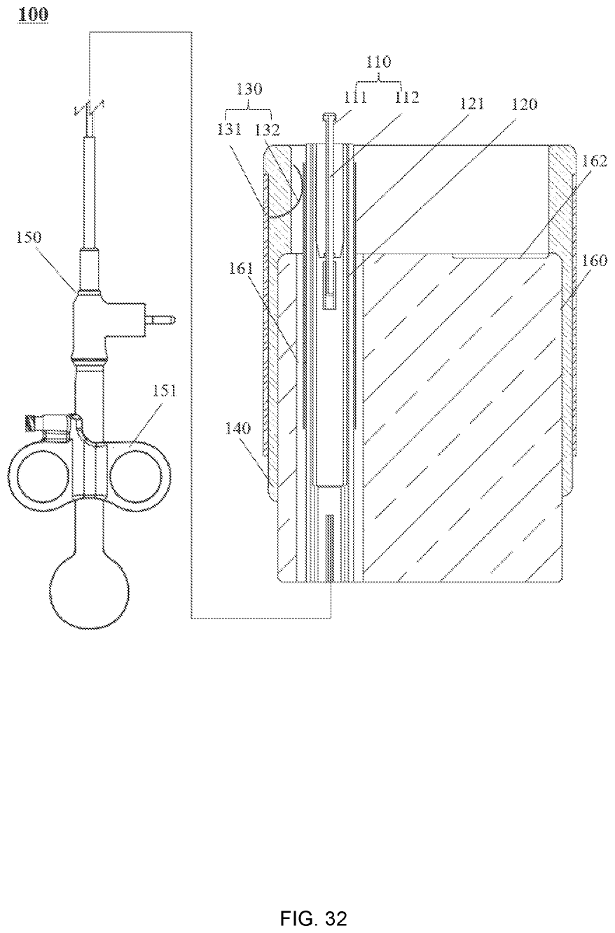

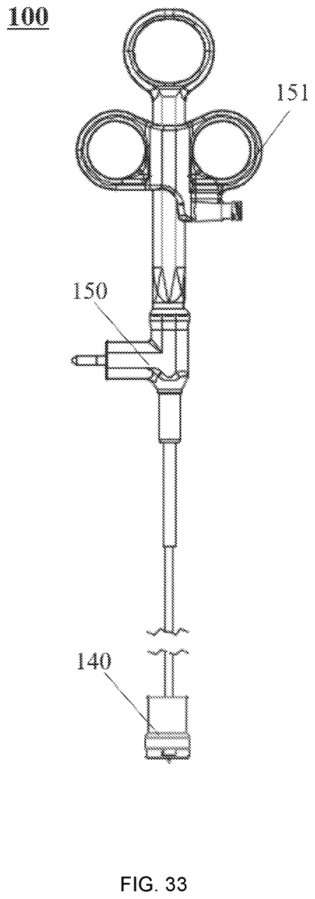

FIG. 31 is a schematic diagram illustrating an exemplary structure of a treatment apparatus for an endoscope according to some embodiments of the present disclosure; FIG. 32 is a schematic diagram illustrating an exemplary structure of a treatment apparatus for an endoscope cooperating with an endoscope according to some embodiments of the present disclosure; FIG. 33 is a schematic diagram illustrating an exemplary structure of an expandable frame mounted on the treatment apparatus for an endoscope according to some embodiments of the present disclosure; FIG. 34 is a schematic diagram illustrating an exemplary structure of an expandable frame and a sheath cooperating with an endoscope, according to some embodiments of the present disclosure; FIG. 35 is a schematic diagram illustrating another exemplary structure of an expandable frame and a sheath cooperating with an endoscope, according to some embodiments of the present disclosure; FIG. 36 is a schematic diagram illustrating an exemplary working simulation of an expandable frame and a sheath cooperating with an endoscope according to some embodiments of the present disclosure.

As shown in FIGS. 31-36, the treatment apparatus 100 for an endoscope may include a first electrode 110 and a sheath 120.

The first electrode may be used to conduct a current with a relatively high frequency to perform a clinical manipulation of a tissue (e.g., an endoscopic surgery). In some embodiments, the first electrode 110 may include an electrical treatment part 111 and an operating wire 112.

The electrical treatment part may be a component used to perform a clinical manipulation of a tissue (e.g., a human tissue). For example, the electrical treatment part may include an electrical trap, electrocoagulation forceps, electrical biopsy forceps, an incisional knife, etc. In some embodiments, the electrical treatment part 111 may be used to perform a clinical manipulation of a human tissue, including, but is not limited to, an electroshock, an electrothermal, an electrocautery, etc.

The operating wire may be a component used to manipulate the electrical treatment part. In some embodiments, the operating wire 112 may be electrically connected to one of poles of a power supply electrode (e.g., an active electrode) at one end and electrically connected to the electrical treatment part 111 at the other end. The power supply electrode may include an active electrode and a passive electrode, where the active electrode supplies power to the electrical treatment part 111, and the electrical treatment part 111 clinically manipulates a human tissue, and a current enters the human tissue and is then led out and returned to the passive electrode via other component(s) (e.g., a second electrode, a flexible conductive part, etc.), thereby forming a current circuit.

In some embodiments, the operating wire 112 and the electrical treatment part 111 may be an integral conductor to enable a current with a relatively high frequency to be conducted from the active electrode to the electrical treatment part 111 via the operating wire 112; the operating wire 112 and the electrical treatment part 111 may be electrically connected via a conductor based on a removable connection (e.g., a threaded connection, etc.), through which the electrical treatment part 111 may be replaced based on an actual condition of a patient to enable it to meet different treatment needs of different patients.

In some embodiments, an electrical connection between the active electrode and the electrical treatment part 111 may be achieved through the operating wire 112 based on other mean(s). For example, the operating wire 112 itself may not be electrically conductive and a separate wire may be connected between the electrical treatment part 111 and the active electrode for the purpose of the electrical connection.

The electrical connection may be used to conduct a current. In some embodiments, the electrical connection may include, but is not limited to, a connection based on a conductor, a wire, a conduit, or a connection based on a conductive fluid (e.g., a tissue fluid, a secretory fluid, etc.) within a human tissue.

In some embodiments, the operating wire 112 may be threaded into the sheath 120 and the electrical treatment part 111 may extend out from a front end of the sheath 120 for a clinical manipulation of a human tissue. The front end may be understood to be an end away from an operator of the treatment apparatus.

The sheath may be a component capable of being moved within the endoscope. The sheath may be an insulating tube, such as an insulating tube made of an insulating material such as resin. In some embodiments, the sheath 120 may be used to insulate and protect the first electrode 110 to avoid a short circuit.

Further, the sheath 120 may be an insulating hose. In some embodiments, the sheath 120 may be connected to a drive part 150 (with sliding ring 151 provided thereon) external to the body and moved within the endoscope 160 under the control of the drive part 150.

The endoscope may be a medical device used to directly view an internal cavity of a human organ. In some embodiments, the endoscope 160 may include an instrument passage 161. The instrument passage may be used to move the sheath within the endoscope.

In some embodiments, the endoscope 160 may also include a lens 162. The lens may be used to obtain an image of the internal cavity of the human organ (e.g., an image of a lesion region, etc.) and transmit it to a display device (not shown in the figures). The operator (e.g., a physician) may perform a corresponding operation based on the image of the internal cavity of the human organ.

In some embodiments, the surface of the sheath 120 is provided with the bendable flexible conductive part 121.

The bendable flexible conductive part may mean that the flexible conductive part 121 may be bent at multiple (e.g., any angles) angles. Exemplarily, the sheath 120 may be bent by 30°, 60°, 90°, 180°, 210°, etc., depending on actual needs as the flexible conductive part 121 is moved through the instrument passage 161 of the endoscope 160, based on the scenario of the use of the treatment apparatus 100 for an endoscope at different positions within a human tissue.

The flexible conductive part may be a bendable structure provided on the surface of the sheath and capable of being used to conduct a current. In some embodiments, the bendable flexible conductive part 121 may be implemented in a variety of ways. For example, it may be achieved by spraying a conductive material (e.g., a conductive paint, etc.) on the surface of the sheath 120 such that a conductive material layer covers at least a portion of the surface of the sheath 120.

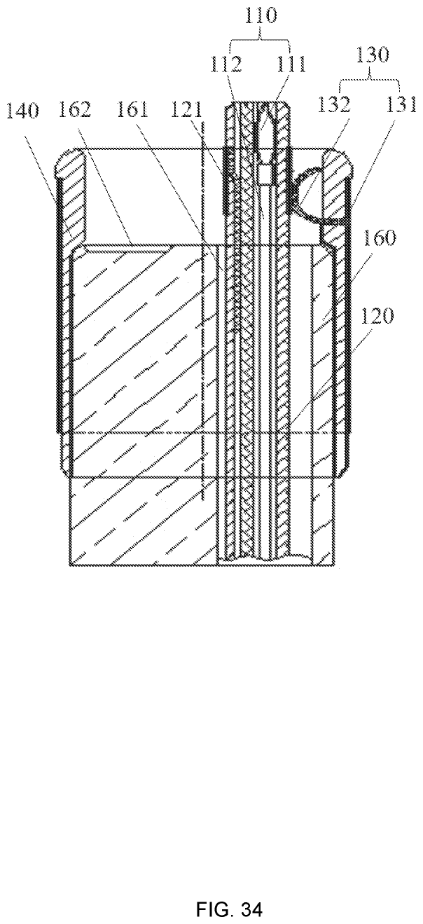

In some embodiments, the flexible conductive part 121 may be in the form of a tube, and the flexible conductive part 121 may have a gap to facilitate a bending of the flexible conductive part, and the surface of the sheath may be connected to the external air of the flexible conductive part through the gap, so as to achieve a bendable function of the flexible conductive part 121. For more information about the flexible conductive part and the gap, please refer to FIGS. 37-39 and their related descriptions.

As shown in FIGS. 33-34, the treatment apparatus 100 for an endoscope may further include a second electrode 130 and an expandable frame 140.

The second electrode may be a component mounted to the endoscope for drawing a current out of a body tissue. In some embodiments, the second electrode 130 may include a first conductive part 131 and a sliding contact part 132 electrically connected to the first conductive part 131.

The first conductive part may be a component for contacting a human tissue to conduct a current from the human tissue to the sliding contact part.

As shown in FIG. 34, the first conductive part 131 may be provided at a periphery of the expandable frame 140 for a direct contact with the human tissue to enable drawing the current out of the human tissue. For more information about the expandable frame, please refer to the following descriptions of the expandable frame.

In some embodiments, the first conductive part 131 may be conductive based on a variety of ways. For example, the first conductive part 131 may be a metal plating, including but not limited to a silver plating, a copper plating, etc.; as another example, the first conductive part 131 may be a conductive plastic or a conductive rubber, including but not limited to a conductive silicone, etc.

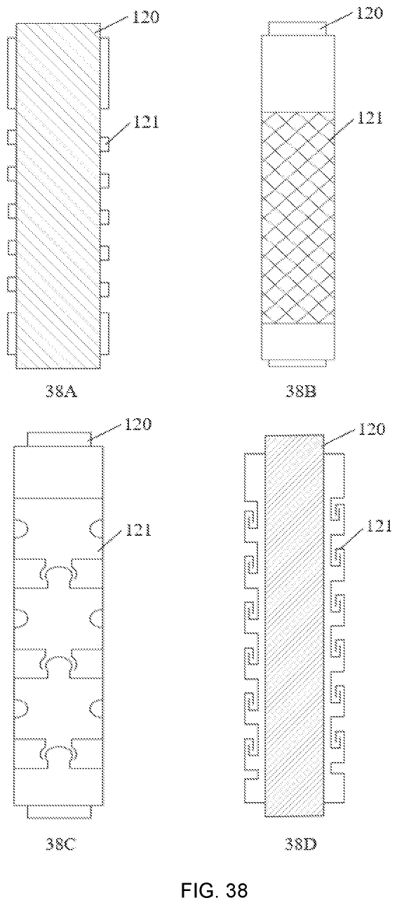

In some embodiments, the first conductive part 131 may include a metal conductive layer 1311. For more information about the first conductive part, please refer to FIGS. 40-42 and their related descriptions.

The sliding contact part may be a component that is electrically connected to the first conductive part for conducting the current from the first conductive part to the flexible conductive part.

In some embodiments, the sliding contact part 132 may include a conductive material and its structure may include but is not limited to the form of a sheet, a rod, etc. It can be understood that the sliding contact part 132 may be connected electrically to the first conductive part 131 and the flexible conductive part 121 so that the current may be conducted from the first conductive part 131 to the flexible conductive part 121 via the sliding contact part 132, and the present disclosure does not limit the structure of the sliding contact part 132.

In some embodiments, the sheath 120 may be threaded into the endoscope 160, with the sliding contact part 132 in contact with and electrically connected to the flexible conductive part 121 when the sheath 120 is in a preset position. Specifically, the sheath 120 may be threaded into the instrument passage 161 of the endoscope 160 and be controlled to move freely in and out of the instrument passage 161 by the drive part 150 (having the sliding ring 151 thereon) connected to the exterior of the body.

In some embodiments of the present disclosure, by having a bendable flexible conductive part on the surface of the sheath, the process may be simplified, the manufacturing cost may be relatively low, the conductive stability may be improved, and it can ensure that the sheath can smoothly pass through the instrument passage at various bending angles, which in turn can make it easier and more convenient for the operator (e.g., a physician) to use in order to shorten the treatment time.

The preset position may refer to a predetermined position where the sheath can reach. In some embodiments, the preset position may be determined based on simulations, experiments, etc.

The preset position may be a specific position, such as 5 mm after the front end of the sheath passes through the endoscope, etc.; alternatively or additionally, the preset position may be a range interval within which the sliding contact part can contact and electrically connect with the flexible conductive part on the sheath when the sheath slides within the range interval.

In some embodiments, the sliding contact part 132 may contact and electrically connect to the flexible conductive part 121 when the sheath 120 is in the preset position, thereby allowing for the sequential drawing of the current from the human tissue through the first conductive part 131, the sliding contact part 132, and the flexible conductive part 121.

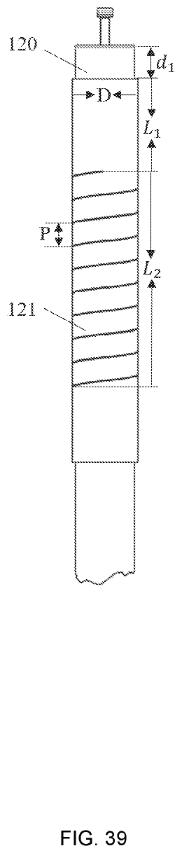

For more information about the sliding contact part, please refer to FIGS. 40-41 and their related descriptions.

The expandable frame may be a component used to maintain a proper endoscopic field of view to aid in the clinical operation. A structure of the expandable frame is not limited and may be straight, stepped cylindrical, conical, etc. In some embodiments, the structure of the expandable frame 140 may be determined based on experiments, simulations, etc.

As shown in FIG. 34, the expandable frame 140 may be used to be snapped onto the outside of the endoscope 160. Preferably, the expandable frame 140 may be snapped onto an apex of the endoscope 160 (i.e., an end of the endoscope away from the operator) to allow the endoscope 160 to avoid interference, focus quickly, and provide a clearer view, which in turn may allow for easier and more precise clinical operation.

In some embodiments, the expandable frame 140 may be made of a flexible insulating material to accommodate the endoscopes 160 of different diameters. In some embodiments, the periphery of the expandable frame 140 may be provided with the first conductive part 131 for the direct contact with the human tissue to reduce the path of current flow through the human body in order to quickly draw the current out of the human tissue.

As shown in FIG. 35, the expandable frame 140 may be made of a flexible conductive material (e.g., a conductive silicone, etc.), i.e., the expandable frame 140 may be in direct contact with the human tissue to draw the current out of the human tissue.

For more information about the expandable frame and the first conductive part, please refer to FIGS. 40-41 and their descriptions.

As shown in FIG. 36, before starting a clinical operation (e.g., an endoscopic surgery), the expandable frame 140 with the first conductive part 131 is attached to the apex of the endoscope 160, and then the sheath 120 with the electrical treatment part 111 (e.g., an electrical trap 1111) is inserted into the instrument passage 161 of the endoscope 160, and then the endoscope 160 is inserted into a human body cavity (e.g., a digestive tract, etc.) while the lens 162 of the endoscope 160 obtains and transmits back image(s) of the internal cavity of the human organ. If the endoscope 160 reaches a lesion region, the operator may operate the sliding ring 151 on the drive part 150 so that the electric trap 1111 is placed over a tissue to be removed, while the first conductive part 131 is pressed against the tissue on a side wall to fully contact with the human tissue. Finally, the power supply electrode is turned on, and the current flows back to the passive electrode of the power supply electrode through the active electrode of the power supply electrode, the operating wire 112, the electric trap 1111, the tissue to be removed, the first conductive part 132, the sliding contact part 132, the flexible conductive part 121, and other parts, thereby forming a current circuit, and generating high heat at a contact part between the electric trap 1111 and the tissue to be removed, so as to remove the tissue to be removed.

In some embodiments of the present disclosure, the treatment apparatus for an endoscope may be provided with a flexible conductive part based on the surface of the sheath, which enables the sheath to move freely in the instrument passage at various angles of the endoscope and makes the apparatus more convenient to use; and by providing an expandable frame on the endoscope and a first conductive part on the periphery of the endoscope, the first conductive part can be brought into full contact with the human tissue, and when the sheath is located in the preset position, the sliding contact part is in contact with and electrically connected to the flexible conductive part, so that the current in the human tissue can be drawn out quickly without flowing through the whole body, which can appropriately increase the current intensity and thus increase the speed of clinical operation (e.g., a cutting speed etc.).

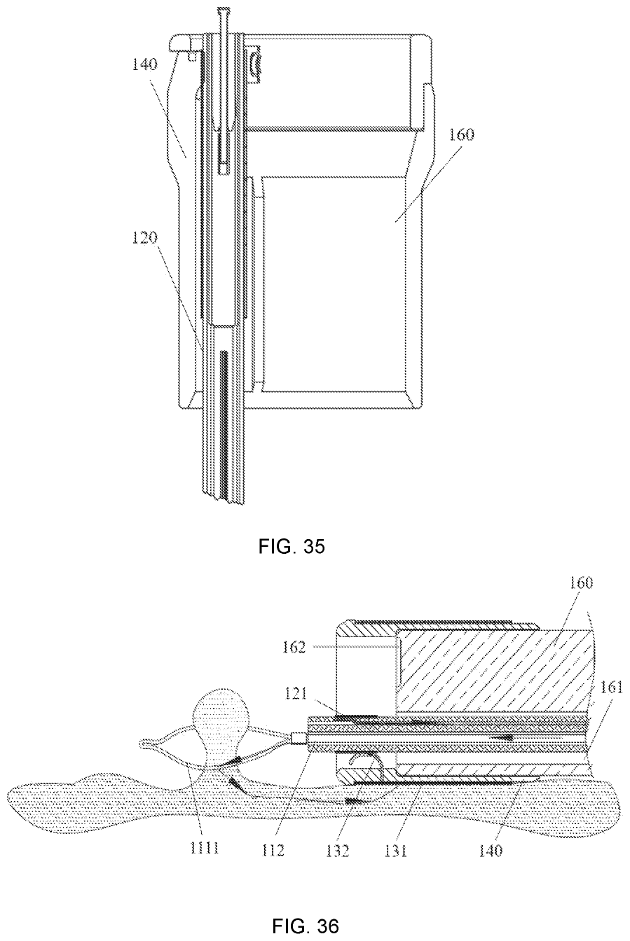

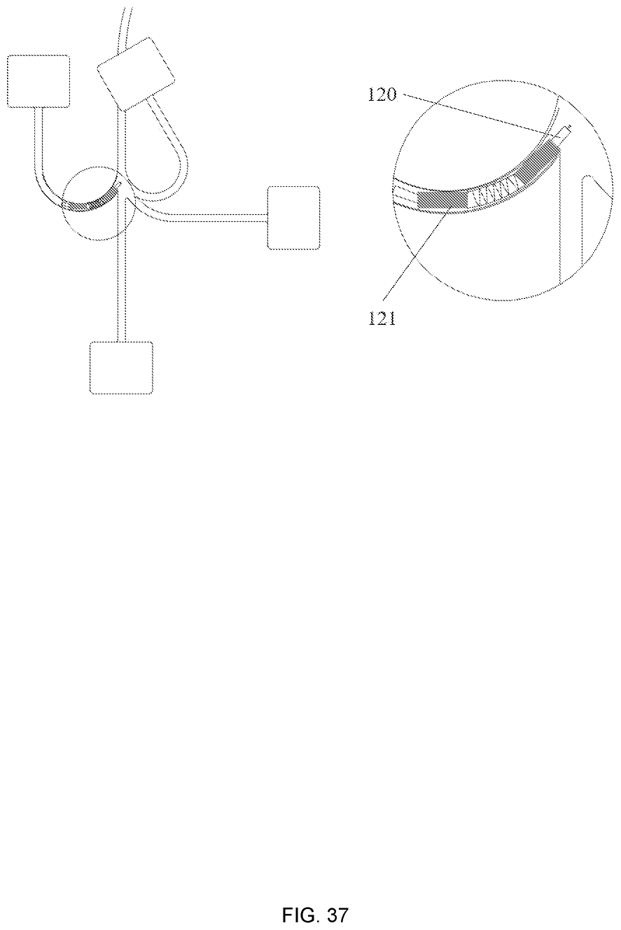

FIG. 37 is a schematic diagram illustrating an exemplary application of a flexible conductive part according to some embodiments of the present disclosure; FIG. 38 is a schematic diagram illustrating an exemplary structure of a bent section of a flexible conductive part according to some embodiments of the present disclosure; FIG. 39 is a schematic diagram illustrating an exemplary structure of a flexible conductive part according to some embodiments of the present disclosure.

As shown in FIGS. 37-39, the flexible conductive part 121 may include a metal member containing a gap.

The metal member may refer to a metal structure provided on the surface of the sheath for conducting a current. In some embodiments, the metal member may include one or more consecutive metal members. The consecutive metal members may be understood as metal articles in each of which the machined and formed parts are formed as one piece.

In some embodiments, the gap may allow the surface of the sheath to be partially exposed. The exposed area may be determined based on different structures of the one or more consecutive metal members.

In some embodiments, the gap may be formed based on the one or more consecutive metal members. Further, the gap may be formed based on the different structures formed by the one or more consecutive metal members. In some embodiments, as shown in FIG. 38A, the flexible conductive part 121 may include a spiral structured metal member, and the spiral structured metal member may form a spiral slit-like gap. In some embodiments, as shown in FIG. 38B, the flexible conductive part 121 may include a woven structured metal member, and the woven structured metal member may form a mesh-like gap. In some embodiments, as shown in FIG. 38C, the flexible conductive part 121 may include a serpentine structured metal member that includes multiple consecutive metal members (i.e., multiple bone joints); the serpentine structured metal member may form a pore-like gap. In some embodiments, as shown in FIG. 38D, the flexible conductive part 121 may include a stack structured metal member that includes multiple consecutive metal members (i.e., multiple stacked parts); the stack structured metal member may form a slit-like gap.

In some embodiments, the gap may include a gap formed between interrupted surfaces of the one or more consecutive metal members, and the gap may be spirally provided in an axial direction of the sheath 120. The interrupted surfaces may be understood as a surface on which one of internal structures of the metal member is opposite to that of another internal structure; or a surface on which one metal member of multiple metal members is opposite to another metal member.

In some embodiments, as shown in FIG. 39, the flexible conductive part 121 may include a single spiral set consecutive metal member. A middle portion of the consecutive metal member may be spirally wound on the surface of the sheath, with a spiral slit formed between the interrupted surfaces of two adjacent wound parts of the wound consecutive metal member. In some embodiments, the flexible conductive part 121 may include two consecutive metal members (not shown in the figure) set in a double spiral, with the two consecutive metal members spirally wound on the surface of the sheath, and a spiral slit formed between the interrupted surfaces of the two consecutive metal members after winding. In some embodiments, the spirally set consecutive metal members may be formed by curling a metal sheet. In some embodiments, the spirally set consecutive metal members may be formed by a metal tube cutting (e.g., laser cutting). Since the spirally set consecutive metal members are a monolithic part, the conductive effect can be effectively ensured; at the same time, a spiral-shaped slit can be formed to enable the consecutive metal members to have good bendability.

In some embodiments, a pitch P of the spirally set slit may not be too large or too small. If the pitch P is too large, it may affect the bendability of the flexible conductive part 121; if the pitch P is too small, it may affect the electrical conductivity of the flexible conductive part 121. In some embodiments, the pitch P of the spirally set slit may be 0.1 to 2 mm. In some embodiments, the pitch P of the spirally set slit may be 0.3 to 1.5 mm. In some embodiments, the pitch P of the spirally set slit may be 0.5 to 1 mm.

In some embodiments, a width of the gap in the metal member along the axial direction of the sheath 120 may be less than a length of the conductive structure that cooperates with the flexible conductive part 121 extending along the axial direction of the sheath 120.

In some embodiments, the conductive structure may refer to a structure that cooperates with the flexible conductive part 121 for conducting the current. In some embodiments, the conductive structure may include the sliding contact part 132, where the width of the gap in the metal member along the axial direction of the sheath 120 is less than the length of the sliding contact part 132 extending along the axial direction of the sheath 120.

In some embodiments of the present disclosure, by making the width of the gap of the metal member along the axial direction of the sheath smaller than the length of the conductive structure extending along the axial direction of the sheath, it is possible to avoid the conductive structure being stuck in the gap due to the gap of the metal member being too large, so that a stable electrical connection between the flexible conductive part and the conductive structure can be ensured so that the current in the human tissue can be drawn out smoothly and damage to the human body can be avoided.

In some embodiments, the width of the gap of the metal member along the axial direction of the sheath 120 may be greater than or equal to a wall thickness of the metal member.

The wall thickness of the metal member may refer to a thickness of the metal member in a radial direction along the sheath. By limiting the width of the gap of the metal member along the axial direction of the sheath to be greater than or equal to the wall thickness of the metal member, the metal member may be made easier to bend, which may effectively ensure a bendable function of the flexible conductive part 121, which in turn may enable the sheath 120 to pass smoothly through the instrument passage 161 of the endoscope 160 at various bending angles, which is conducive to saving operation time and improving the patient treatment experience.

In some embodiments, as shown in FIG. 39, the flexible conductive part may include a bent section and a straight section.

The bent section may refer to a bendable portion of the flexible conductive part. In some embodiments, the bent section is used to implement the bendable function of the flexible conductive part 121 to allow the sheath 120 wrapped in the flexible conductive part 121 to pass smoothly through the instrument passage 161 of the endoscope 160 at various bending angles.

The straight section may refer to a straight portion of the flexible conductive part. In some embodiments, the straight section may be electrically connected to the bent section.

In some embodiments, the electrical connection between the straight section and the bent section may be implemented based on a variety of approaches. For example, the straight section and the bent section may be a one-piece structure to form an electrical connection; as another example, the straight section and the bent section may be electrically connected by welding, etc.

In some embodiments, a length of the straight section may be related to a diameter of the straight section. In some embodiments, in order to ensure that the flexible conductive part 121 is bendable at an angle such that the sheath 120 wrapped by the flexible conductive part 121 can pass smoothly through the instrument passage 161 of the endoscope 160 at various bending angles, the length of the straight tube segment may be less than or equal to 6 times the diameter of the straight section.

In some embodiments, the straight section(s) and the bent section(s) may be combined to form the flexible conductive part 121, in which the count of the straight section(s) and the bent section(s) and/or the form of combination thereof are not limited. For example, the flexible conductive part 121 may be formed based on a combination of one straight section and one bent section; or, as another example, the flexible conductive part 121 may be formed based on three straight sections and two bent sections, in the form of a cross combination of straight sections and bent sections, etc. In some embodiments, the flexible conductive part 121 may include two straight sections and two bent sections.

In some embodiments, the flexible conductive part 121 may include two straight sections, and the bent section is connected between the two straight sections. In some embodiments, in order to ensure the bending ability of the bent section and enhance the bendability of the flexible conductive part 121, the length of the bent section may be greater than or equal to two times the diameter of the bent section. In some embodiments, the diameter of the bent section may be equal to the diameter of the straight section.

In some embodiments, if the flexible conductive part 121 includes a bent section connected between two straight sections, the length of the straight section located at a front end (i.e., the straight section away from the operator of the treatment apparatus) may be greater than the length of the straight section located at a rear end (i.e., the straight section near the operator of the treatment apparatus) to better match the use of the expandable frame 140. In some embodiments, the exact length of the straight section located at the front end and that of the straight section located at the rear end may be determined based on actual circumstances.

In some embodiments of the present disclosure, by the design of the relationship between the length of the bent section and its diameter, it is possible to ensure not only the bendable angle of the flexible conductive part to fully ensure its over-bending performance, but also to guarantee the conductive performance between the flexible conductive part and the sliding contact part and between the straight section and the bent section of the flexible conductive part.

As shown in FIGS. 37-39, the front end of the sheath 120 extends out from the front end of the flexible conductive part 121. In some embodiments, a distance that the front end of the sheath 120 extends out from the front end of the flexible conductive part 121 may not be limited in the present disclosure, and it may be determined based on actual circumstances. For example, extending lengths or distances for different electrical treatment parts 111 (e.g., the electrical trap, the electrical coagulation clamp, etc.) may be different.

In some embodiments of the present disclosure, by extending the front end of the sheath beyond the front end of the flexible conductive part, the electrical treatment part provided at the front end of the sheath may be kept at a distance from the flexible conductive part to avoid a short circuit (i.e., shorting out the first conductive part 131) caused by a liquid backflow (e.g., tissue fluid, etc.) into the flexible conductive part while the electrical treatment part is under clinical operation.

As shown in FIG. 39, a first distance d¹ may exist between the front end of the flexible conductive part 121 and the front end of the sheath 120.

The first distance may refer to a distance between the front end of the flexible conductive part and the front end of the sheath.

In some embodiments, a range of the first distance d¹ may be from 0.5 mm to 1.5 mm. In some embodiments, the range of the first distance d¹ may be other range intervals, which may be determined based on experiments, simulations, etc.

In some embodiments, the first distance d¹ may be equal to a second distance d₂ that exists between a front end of the sliding contact portion 132 and a front end of the transparent straight frame 141 of the expandable frame 140 to avoid a short circuit due to a liquid backflow (e.g., tissue fluid) during use of the apparatus, which in turn may ensure the safety of the apparatus during use and may protect the apparatus to a certain extent.

For more information about the transparent straight frame and the second distance of the expandable frame, please refer to FIG. 41 and their related descriptions.

In some embodiments of the present disclosure, by setting the first distance between the front end of the flexible conductive part and the front end of the sheath, and limiting the range of the first distance, the flexible conductive part can be better used with the expandable frame to avoid a short circuit due to a liquid backflow during the use of the apparatus, thus ensuring the safety of the apparatus when it is used; at the same time, it can also protect the apparatus from damage due to the short circuit.

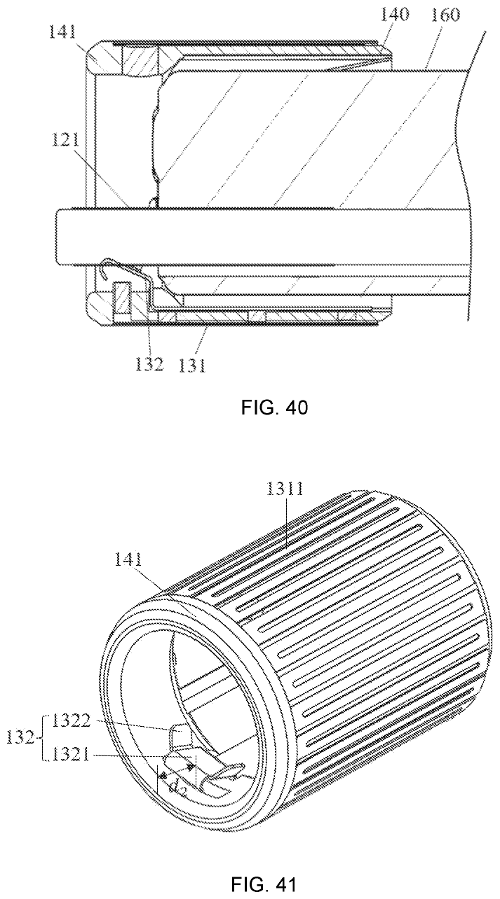

FIG. 40 is a schematic diagram illustrating an exemplary structure of an expandable frame and a first conductive part cooperating with a sliding contact part according to some embodiments of the present disclosure; FIG. 41 is a schematic diagram illustrating another exemplary structure of an expandable frame and a first conductive part cooperating with a sliding contact part according to some other embodiments of the present disclosure; FIG. 42 is a schematic diagram illustrating an exemplary structure of a metal conductive layer according to some embodiments of the present disclosure.

As shown in FIGS. 40-42, the first conductive part 131 may include a metal conductive layer 1311 of variable diameter provided on the periphery of the expandable frame 140.

In some embodiments, the variable diameter may mean that a diameter of the metal conductive layer 1311 provided at the periphery of the expandable frame 140 may vary based on a size of the diameter of the endoscope 160 so that it may be applicable to the endoscope 160 with various diameters.

In some embodiments, the metal conductive layer 1311 may be made of one or more conductive materials, such as copper, silver, etc. In some embodiments, the metal conductive layer 1311 may be a thin layer to reduce the volume thereof. Further, the metal conductive layer 1311 may be a smooth, thin layer to reduce harm to the human body. In some embodiments, a structure of the metal conductive layer 1311 is not limited. For example, the metal conductive layer 1311 may be a folded structure, a grid strip structure, etc.

In some embodiments, the metal conductive layer 1311 may be a bow shape structure.

As shown in FIGS. 41-42, the bow shape structure is a structure formed by bending the metal sheet at a preset width and connecting the end and the beginning thereof. The preset width may be a predetermined bending width, which may be determined based on actual needs. For example, the preset width may be determined based on a width of the metal conductive layer 1311, etc.

It can be understood that as the diameter of the endoscope 160 increases, the bow shape structure may be extended along a circumferential direction of the expandable frame 140 so that the diameter of the metal conductive layer 1311 increases accordingly to accommodate the endoscope and expandable frame with various diameters.

In some embodiments of the present disclosure, by using the bow shape structure to achieve the function of the variable diameter of the metal conductive layer, the metal conductive layer may have a simple structure, easy to be processed, and may have a low manufacturing cost.

In some embodiments of the present disclosure, by providing the metal conductive layer of a variable diameter around the periphery of the expandable frame, it can not only ensure a contact area between the metal conductive layer and the human tissue to avoid the risk of overheating burns that may occur due to a small contact area; but also enable the expandable frame to be adapted to the endoscopes of various diameters, which in turn can improve the applicability and practicality of the expandable frame.

As shown in FIGS. 40-41, the sliding contact part 132 may also include an inwardly protruding metal shrapnel 1321, which may be electrically connected to the first conductive part 131.

The metal shrapnel may be a thin sheet of metal having a certain degree of elasticity. In some embodiments, the metal shrapnel 1321 may be electrically connected to the first conductive part 131 based on a variety of ways. For example, the metal shrapnel 1321 may be a one-piece structure with the metal conductive layer 1311 of the first conductive part 131 to achieve the electrical connection; as another example, the metal shrapnel 1321 is a split structure with the metal conductive layer 1311 of the first conductive part 131, and the metal shrapnel 1321 is electrically connected to the first conductive part 131 through direct contact; as another example, the metal shrapnel 1321 may be electrically connected to the metal conductive layer 1311 of the first conductive part 131 by other conductors. The metal shrapnel 1321 may be electrically connected by contacting the metal conductive layer 1311 of the first conductive part 131 with other conductors, etc.

In some embodiments, when the sheath 120 is moved to the preset position, the sliding contact part 132 may contact and form an electrical connection with the flexible conductive part 121 on the sheath 120 through the inwardly protruding metal shrapnel 1321, thereby enabling a current conduction.

In some embodiments, a structure and a size of the metal shrapnel 1321 are not limited. For example, a portion of the metal shrapnel 1321 may be electrically connected through the expandable frame 140 to the metal conductive layer 1311 set on the periphery of the expandable frame 140, while another portion of the metal shrapnel 1321 may be an arc sheet adapted to the diameter size of the sheath 120, etc.

As shown in FIG. 41, two lugs 1322 are provided on both sides of the metal shrapnel 1321, and the two lugs 1322 are electrically connected to the metal shrapnel 1321.

The lugs may be an auxiliary structure for restraining the sheath. In some embodiments, a manner in which the lugs 1322 are provided may be related to the size of the diameter of the sheath 120. Exemplarily, if the diameter of the sheath 120 is relatively large, an angle between the two lugs 1322 may be relatively large, i.e., a space enclosed by the two lugs 1322 may be relatively large; if the diameter of the sheath 120 is relatively small, an angle between the two lugs 1322 may be relatively small, i.e., a space enclosed by the two lugs 1322 may be relatively small.

In some embodiments, the lugs 1322 and the metal shrapnel 1321 may be configured as one piece or separate pieces.

In some embodiments, the two lugs 1322 may form a semi-enveloping structure with the metal shrapnel 1321 against the flexible conductive part 121 for restraining the sheath 120.

Specifically, when the sheath 120 moves forward and through the semi-enveloping structure formed by the metal shrapnel 1321 and the two lugs 1322 to the preset position, the inwardly protruding metal shrapnel 1321 and the lugs 1322 provided on both sides of the metal shrapnel 1321 may be in sufficient contact with the flexible conductive part 121 on the sheath 120 to achieve a stable electrical connection.

In addition, the presence of the semi-enveloping structure can restrain the sheath 120 to a certain extent to avoid its swinging from side to side, thereby improving the stability of the electrical processing part 111 in the performance of the clinical operation.

In some embodiments of the present disclosure, the metal shrapnel is electrically connected to the first conductive part, and when the sheath is moved to the preset position, the inwardly protruding metal shrapnel may fully contact with the flexible conductive part on the sheath, and thus, a stable electrical connection may be formed between the first conductive part, the metal shrapnel and the flexible conductive part, which is conducive to the rapid drawing out of the current in the human tissue to ensure the safety of the apparatus in use and to avoid damage to the human body.

As shown in FIGS. 40-41, the sliding contact part 132 may be set on the expandable frame 140, and the expandable frame 140 may include a transparent straight frame 141 set at a front end of the expandable frame

The transparent straight frame may refer to a transparent portion of a front portion of the expandable frame. In some embodiments, the transparent straight frame 141 may be made of a transparent flexible insulating material, which may enable a more open and clear view of the endoscope 160, thereby allowing the operator to observe the lesion in more detail, and thus allowing better clinical operation.

As shown in FIG. 41, the second distance d₂ may exist between the front end of the sliding contact part 132 and a front end of the transparent straight frame 141.

The second distance may be a distance between the front end of the sliding contact part and the front end of the transparent straight frame. In some embodiments, the second distance d₂ may range from 0.5 mm to 1.5 mm. In some embodiments, a range of the second distance d₂ may be other range intervals, which may be determined based on experiments, simulations, etc.

In some embodiments, the first distance d¹ exists between the front end of the flexible conductive part 121 and the front end of the sheath 120, and the first distance d¹ may be equal to the second distance d₂, i.e., d₁=d₂. By setting the first distance d¹ existing between the front end of the flexible conductive part 121 and the front end of the sheath 120, and the second distance d₂ existing between the front end of the sliding contact part 132 and the front end of the transparent straight frame 141, the flexible conductive part 121 can be better used with the expandable frame 140 to avoid the short circuit caused by the a liquid backflow (e.g., tissue fluid) during the use of the apparatus, which can ensure the safety of the apparatus and protect the apparatus to a certain extent.

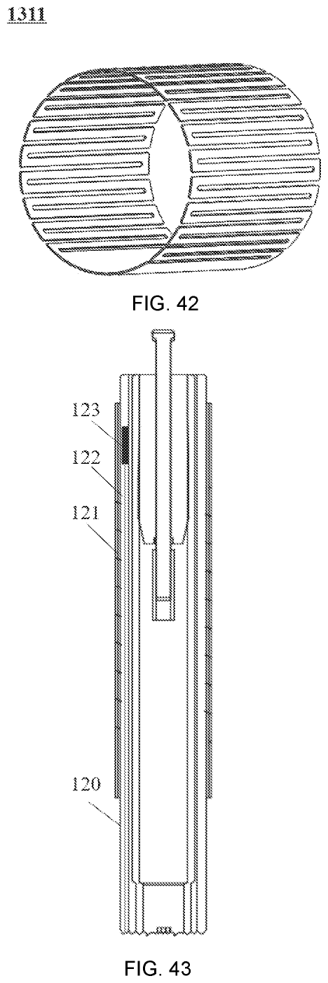

FIG. 43 is a cross-sectional diagram illustrating the sheath and the flexible conductive part according to some embodiments of the present disclosure.

As shown in FIG. 43, the sheath 120 is provided with a return conductor 122, which passes through the sheath 120 and is electrically connected to the flexible conductive part 121.

The return conductor may be a component used to conduct the current back to the passive electrode. In some embodiments, a material of the return conductor 122 may be molybdenum wire, silver wire, etc., which is not limited.

In some embodiments, an electrical connection of the return conductor 122 to the flexible conductive part 121 may be achieved based on a variety of ways. For example, the return conductor 122 may be electrically connected by a direct contact with the flexible conductive part 121; as another example, the return conductor 122 may be electrically connected by other conductor(s) contacting the flexible conductive part 121, etc.

In some embodiments, the sheath 120 is provided with an opening (not shown in the figures) through which the return conductor 122 passes, and a metal restriction tube 123 is provided at the opening, and the flexible conductive part 121 is connected to the metal restriction tube 123.

The opening may be a through-hole in the sheath for the return conductor to pass through. The opening may be a circular hole, an oval or other shaped hole, and a shape of the opening may be not limited. In some embodiments, a size of the opening may be determined based on actual circumstances. For example, the size of the opening may be determined based on a size of the metal restriction tube 123.

The metal restriction tube may be a component used to connect the return conductor to the flexible conductive part. In some embodiments, a material of the metal restriction tube 123 may be a conductive metal such as copper, silver, etc., which is not limited.

In some embodiments, a connection of the metal restriction tube 123 to the flexible conductive part 121 may be a fixed connection. The fixed connection may include a non-detachable connection (e.g., welded, etc.) or a detachable connection (e.g., snap connection, etc.).

In some embodiments, based on the metal restriction tube 123 being connected to the flexible conductive part 121, the return conductor 122 may be electrically connected to the flexible conductive part 121 so that the current in the human tissue can be returned to the passive electrode via the first conductive part 131, the sliding contact part 132, the flexible conductive part 121, the metal restriction tube 123, and the return conductor 122.

In some embodiments of the present disclosure, the connection stability of the electrical connection can be better ensured by using the metal restriction tube to electrically connect the return conductor to the flexible conductive part.

In some embodiments of the present disclosure, by providing the return conductor inside the sheath and the return conductor passing through the sheath to achieve a stable electrical connection with the flexible conductive part, the current in the human tissue can be smoothly and quickly returned to the passive electrode, thus ensuring the smooth operation.

Some embodiments of the present disclosure provide a treatment apparatus for an endoscope, the apparatus includes a first electrode, the first electrode includes an electrical treatment part and an operating wire; a sheath, a surface of the sheath is provided with a bendable flexible conductive part; wherein the operating wire is threaded into the sheath, the electrical treatment part extends from a front end of the sheath, and the front end of the sheath extends from a front end of the flexible conductive part.

Some embodiments of the present disclosure provide an expandable frame used to be snapped onto an outside of the endoscope, with a first conductive part around a periphery of the expandable frame, the first conductive part includes a metal conductive layer of variable diameter.

Possible beneficial effects of the embodiments of the present disclosure include, but are not limited to: (1) based on the surface of the sheath, the bendable flexible conductive part is provided, which is simple, has low manufacturing cost and good conductive stability, and can allow the sheath to move freely in the instrument passage of the endoscope at various curved angles, thus making the apparatus more convenient to use; (2) based on the front end of the flexible conductive part and the front end of the sheath, the first distance is provided, and the range of the first distance is set so that the flexible conductive part can be better used with the expandable frame to avoid the short circuit due to a liquid backflow during the use of the apparatus, thus ensuring the safety of the apparatus in use, and also protecting the apparatus from damage due to the short circuit; (3) based on the provision of the expandable frame over the endoscope and the first conductive part on the periphery of the expandable frame, the first conductive part can be brought into full contact with the human tissue, and when the sheath is located at the preset position, the sliding contact part makes contact with the flexible conductive part and forms a stable electrical connection, which can realize the rapid drawing out of the current from the human tissue without flowing through the whole body, and thus can reduce the risk of causing damage to the human body; moreover, it can appropriately increase the current intensity and thus the speed of the clinical operation (e.g., cutting speed, etc.). It should be noted that different embodiments may produce different beneficial effects, and in different embodiments, the possible beneficial effects may be any one or a combination of the above, or any other beneficial effect that may be obtained.

The technical features in the foregoing embodiments may be randomly combined. For simplicity of description, all possible combinations of the technical features in the foregoing embodiments are not described. However, it should be considered that these combinations of technical features fall within the scope recorded in the specification provided that these combinations of technical features do not have any conflict.

The foregoing embodiments only describe several implementations of the present invention, and their description is specific and detailed, but cannot therefore be understood as a limitation to the patent scope of the present invention. It should be noted that a person of ordinary skill in the art may further make variations and improvements without departing from the conception of the present invention, and these all fall within the protection scope of the present invention. Therefore, the patent protection scope of the present invention should be subject to the appended claims.

What is claimed is:

1. A treatment apparatus for an endoscope, comprising:
a first electrode, the first electrode including an electrical treatment part and an operating wire;
a sheath, a surface of the sheath being provided with a bendable flexible conductive part, the flexible conductive part being in the form of a tube, the flexible conductive part having a gap formed in the flexible conductive part to facilitate a bend of the flexible conductive part, the surface of the sheath being communicated with an external air of the flexible conductive part through the gap;
wherein the operating wire is threaded into the sheath and the electrical treatment part extends out from a front end of the sheath.

2. The treatment apparatus of claim 1, wherein the flexible conductive part includes a metal member containing the gap, the metal member including one or more consecutive metal members, the gap between the one or more consecutive metal members being formed such that the surface of the sheath is partially exposed.

3. The treatment apparatus of claim 2, wherein the gap includes a slit formed between interrupted surfaces of the one or more consecutive metal members, the slit being spirally provided in an axial direction of the sheath.

4. The treatment apparatus of claim 3, wherein a pitch of the spirally provided slit is 0.1 to 2 mm.

5. The treatment apparatus of claim 1, wherein a width of the gap along an axial direction of the sheath is less than a length of a conductive structure cooperating with the flexible conductive part extending along the axial direction of the sheath.

6. The treatment apparatus of claim 1, wherein the flexible conductive part includes a metal member containing the gap, a width of the gap along an axial direction of the sheath being greater than or equal to a wall thickness of the metal member.

7. The treatment apparatus of claim 1, wherein the flexible conductive part includes a bent section and a straight section, the straight section being electrically connected to the bent section, a length of the straight section being less than or equal to six times a diameter of the straight section.

8. The treatment apparatus of claim 7, wherein the flexible conductive part includes two straight sections, the two straight sections including the straight section and a second straight section, the bent section being connected between the two straight sections, a length of the bent section being greater than or equal to two times the diameter of the bent section.

9. The treatment apparatus of claim 1, wherein the front end of the sheath extends out from a front end of the flexible conductive part.

10. The treatment apparatus of claim 9, wherein a first distance between the front end of the flexible conductive part and the front end of the sheath is in a range from 0.5 mm to 1.5 mm.

11. The treatment apparatus of claim 1, further comprising:
a second electrode; and
an expandable frame,
wherein:
the second electrode is used to be mounted on the endoscope,
the second electrode includes a first conductive part and a sliding contact part electrically connected to the first conductive part,
the expandable frame is used to fit on an outside of the endoscope,
the first conductive part is provided on a periphery of the expandable frame;
the sheath is used to be threaded inside the endoscope,
the sliding contact part is in contact with and electrically connected to the flexible conductive part when the sheath is in a preset position.

12. The treatment apparatus of claim 11, wherein the first conductive part includes a metal conductive layer of a variable diameter provided on the periphery of the expandable frame.

13. The treatment apparatus of claim 12, wherein the metal conductive layer is in a bow shape.

14. The treatment apparatus of claim 11, wherein the sliding contact part includes a metal shrapnel protruding inwardly, the metal shrapnel being electrically connected to the first conductive part;
the metal shrapnel is provided with two lugs on both sides of the metal shrapnel, the two lugs being electrically connected to the metal shrapnel, the two lugs and the metal shrapnel forming a semi-enveloping structure for the flexible conductive part.

15. The treatment apparatus of claim 11, wherein the sliding contact part is provided on the expandable frame, the expandable frame includes a transparent straight frame provided at a front end of the expandable frame, and a front end of the sliding contact part has a second distance from a front end of the transparent straight frame.

16. The treatment apparatus of claim 15, wherein the second distance is in a range from 0.5 mm to 1.5 mm.

17. The treatment apparatus of claim 15, wherein a first distance between the front end of the flexible conductive part and the front end of the sheath is equal to the second distance.

18. The treatment apparatus of claim 1, wherein the sheath is provided with a return conductor, the return conductor passing through the sheath and being electrically connected to the flexible conductive part;
the sheath is provided with an opening through which the return conductor passes, the opening being provided with a metal restriction tube, and the flexible conductive part being connected to the metal restriction tube.

19. A treatment apparatus for an endoscope, comprising:
a first electrode, the first electrode including an electrical treatment part and an operating wire;
a sheath, a surface of the sheath being provided with a bendable flexible conductive part;
wherein the operating wire is threaded into the sheath, the electrical treatment part extends out from a front end of the sheath, and the front end of the sheath extends out from a front end of the flexible conductive part.

20. An expandable frame, wherein the expandable frame is provided with a first conductive part on a periphery of the endoscope, the first conductive part including a metal conductive layer of a variable diameter.
